(19) United States
(12) Patent Application Publication
(10) Pub. No.: US 2023/0233498 A1
(43) Pub. Date: Jul. 27, 2023

(54) COMPOSITIONS AND METHODS FOR THE TREATMENT OF COVID-19

(71) Applicant: PROTHIONE, LLC, Franklin, TN (US)

(72) Inventors: Albert B. CRUM, Rhinebeck, NY (US); Laura LILE, Franklin, TN (US)

(21) Appl. No.: 18/002,880

(22) PCT Filed: Jun. 25, 2021
(86) PCT No.: PCT/US2021/039251

§ 371 Date: Dec. 22, 2022

Related U.S. Application Data
(60) Provisional application No. 63/045,028, filed on Jun. 26, 2020.
(60) Provisional application No. 63/093,713, filed on Oct. 19, 2020.

(51) Int. Cl. A61K 31/198; A61K 33/04; A61K 31/122; A61K 45/06; A61P 31/14

(52) U.S. Cl. CPC A61K 31/198; A61K 33/04; A61K 31/122; A61K 45/06; A61P 31/14

(57) ABSTRACT

Embodiments of the present invention relate generally to compositions and methods and uses thereof for preventing, treating or ameliorating the symptoms of Coronavirus (CoV) infections, for example, the novel coronavirus disease 2019 (COVID-19) that is caused by SARS-CoV-2, in a subject in need thereof. Such compositions comprise certain metallothionein analogs, e.g., compositions comprising a glutathione precursor and a selenium source that elevate intracellular glutathione. The methods comprise administering to the subject a composition, e.g., a composition comprising a glutathione precursor and a selenium source, which is capable of inhibiting the intracellular replication and/or infectivity of coronaviruses, particularly the SARS-CoV-2 virus or viral particles thereof.

### TECHNICAL FIELD

The subject matter described herein generally relates to compositions and methods for treating Coronavirus (CoV) infections, for example, the novel coronavirus disease 2019 (COVID-19) caused by severe acute respiratory syndrome coronavirus 2 (SARS-CoV-2).

### BACKGROUND

The severe acute respiratory syndrome coronavirus 2 (SARS-CoV-2), which emerged in December 2019 and causes the novel coronavirus disease 2019 (COVID-19), has rapidly spread across continents and remains a significant threat to public health, health systems, and local and global economies. The virus was previously known as the 2019 novel coronavirus or 2019-nCoV, and is also referred to by the WHO when communicating to the public as “the virus responsible for COVID-19” or “the COVID-19 virus.” The SARS-CoV-2 virus belongs to the family of viruses known as Coronaviruses (CoVs), which are a group of highly diverse, enveloped, positive-sense, and single-stranded RNA viruses that include SARS-CoV-2. The SARS-CoV-2 contains four structural proteins, known as the S (spike), E (envelope), M (membrane), and N (nucleocapsid) proteins. The N protein contain the RNA genomic information. The S, E, and M proteins together create the viral envelope.

Similar to other viruses, SARS-CoV-2 causes infections by going through a viral replication cycle. The replication cycle begins with fusion of the virus with a host cell wherein the S protein is responsible for the initial attachment of the virus to the host cell. After entering into the host cell, the virus releases nucleic acid and forces the cell to replicate the viral genome. Transcription and translation subsequently occurs for protein synthesis and assembly of viral components. The newly formed virus is released from the host cell to extracellular space. The viral load can cause pathogenesis after increasing to certain point. The common symptoms of COVID-19 include among other things, fever, cough and shortness of breath.

COVID-19 appears to be less often fatal than the coronavirus causing severe acute respiratory syndrome (SARS) or Middle East Respiratory Syndrome (MERS). It is more significantly fatal than the seasonal flu. The fatality rate was over two percent according to one study. Nevertheless, COVID-19 has become global pandemic in 2020 after emerging in 2019 and could have severe impacts on the global economy and life. A major challenge in containing the spread of SARS-COV-2 is its person-to-person transmissibility via respiratory droplets and its aerosol and surface stability. More so than at any previous time in modern history, there is an urgent and significant need to rapidly and efficiently decontaminate public and private spaces to prevent the spread of disease. Current methods of disinfection, which include chemical disinfectants and ultraviolet light are labor-intensive and not safe for direct human contact. There is currently no widely-available, cost-effective method to inactivate viruses in spaces where humans are also present.

There are also currently no FDA-approved therapeutics or devices for the treatment of COVID-19. There is a significant unmet need for novel innovations to treat, prevent, or ameliorate the symptoms of COVID-19 caused by SARS COV-2, in order to reduce the severity of disease and secondary complications including progression to pneumonia, acute respiratory distress syndrome, organ injury, septic shock, or death. Given the time required for the testing of newly developed vaccine strategies and the discovery of effective antivirals, alternate approaches to treat, prevent, or ameliorate the symptoms of viral infections where there is no current therapy, for example infections caused by Coronaviruses (CoVs) such as the COVID-19 infection caused by SARS-CoV-2, which can negatively impact viral infection and intracellular replication, are urgently desired.

One approach to enhance discovery of antivirals is to explore the role(s) of the innate immune defenses engaged during the SARS-CoV-2 virus infection. One of the primordial innate defense responses, conserved across nearly every species, is that of the glutathione (reduced form: GSH; oxidized form: GSSG), which comprises part of the essential antioxidant defense system. Glutathione is also the smallest sulfur-containing protein in the cell and serves to reduce reactive oxygen species generated in cells under oxidative stress. It is also involved in the chelation of divalent metal ions.

Although there is some report that glutathione may impact viruses (see, Fraternale et al., Mol Aspects Med, 30(1-2): 99-110, 2009; Fraternale et al., Curr Med Chem, 13(15): 1749-55, 2006; and Fraternale et al., Antiviral Res, 77(2): p. 120-7, 2008), most of these initial reports involve utilization of whole glutathione, often in reduced form (GSH). These in vitro studies have limited applicability in the context of treating or preventing infections caused by Coronaviruses (CoVs) such as the COVID-19 infection caused by SARS-CoV-2 because whole glutathione is poorly transported into cells and therefore provides limited bioavailability.

Oral administration of glutathione to treat various conditions is also largely deemed ineffective and that prodrugs or precursor therapy would be necessary (see, e.g., National Clinical Trials #NCT01251315). Cysteine, or a more bioavailable precursor of cysteine, N-acetyl cysteine (NAC), has been suggested as candidates for precursor therapy. While cysteine and NAC are both, themselves, antioxidants, their presence competes with glutathione for resources in certain reducing (GSH recycling) pathways. Since glutathione is a specific substrate for many redox pathways, the loading of a host with cysteine or NAC may result in less efficient utilization or recycling of glutathione. Thus, cysteine and NAC are not ideal GSH prodrugs. Thus, while GSH may be degraded, and non-physiologically transported as amino acids, there is a physiological barrier to the importation of intact glutathione. As such, these conventional methods fail to provide a reliable and safe means for increasing intracellular GSH levels, especially in the therapeutic context.

Additionally, since glutathione has been shown to increase the vestigiality of the glutathione synthetic machinery via negative feedback inhibition of certain enzymes that are critical to its synthesis (see, e.g., Richman et al., J Biol Chem, 250(4):1422-6, 1975), therapy with whole glutathione may pose unintended side effects. Accordingly, there is an unmet need for new compositions and methods for treating, preventing, or ameliorating symptoms of viral infections where there is no current therapy, for example, infections caused by Coronaviruses (CoVs) such as the COVID-19 infection caused by SARS-CoV-2, which currently have severe impacts on the global economy and life due to the pandemic.

### SUMMARY OF THE INVENTION

This disclosure generally relates to compositions and methods of treating or inhibiting viral infections where there is no current therapy, for example, coronavirus infections such as the COVID-19 infection caused by the SARS-CoV-2 virus or viral particles thereof, by administering to a subject in need thereof an effective amount of a composition that can increase the concentration of intracellular glutathione, such as a composition comprising free form amino acid precursors (FFAAP) of glutathione. Such compositions and methods thereof can particularly inhibit intracellular replication of the SARS-CoV-2 virus or viral particles thereof, or infectivity of the virus or viral particles, or inhibit both intracellular replication and infectivity of the virus or viral particles.

The following aspects and embodiments thereof described and illustrated below are meant to be exemplary and illustrative, not limiting in scope. Embodiments described herein further relate to advantageously treating viral infections where there is no current therapy, for example, infections caused by Coronaviruses (CoVs) such as the COVID-19 infection caused by the SARS-CoV-2 virus or viral particles. For instance, the instant compositions and methods may help eliminate or reduce the SARS-CoV-2 viral load and/or titers in affected subjects, cells and/or tissues.

The compositions and methods described herein further provide for the treatment of viral infections where there is no current therapy, for example, infections caused by Coronaviruses (CoVs) such as the COVID-19 infection caused by the SARS-CoV-2 virus or viral particles, in subjects who need such treatment, e.g., humans and monkeys. Preferably, the compositions and methods described herein are useful for treating a subject diagnosed with COVID-19.

In various embodiments, the present invention relates to a method for preventing or treating a coronavirus infection or for ameliorating symptoms due to coronavirus infection in a subject in need thereof, comprising administering to the subject an effective amount of a composition that increases intracellular glutathione, wherein said administering prevents or treats infection caused by coronavirus or ameliorates symptoms due to coronavirus infection.

In various embodiments, the composition comprises a glutathione precursor and a selenium compound at an amount effective to reduce infectivity of severe acute respiratory syndrome coronavirus 2 (SARS-CoV-2) or viral particles thereof. In various embodiments, the glutathione precursor comprises glycine, L-cystine and a glutamate source. In various embodiments, the glutamate source is glutamine or glutamic acid. In various embodiments, the selenium compound is selenomethionine, selenite, methylselenocysteine or selenium nanoparticles.

In various embodiments, the infectivity of the SARS-CoV-2 virus or SARS-CoV-2 viral particles is reduced by at least about 10%, at least about 15%, at least about 20%, at least about 25%, at least about 30%, at least about 40%, at least about 50%, at least about 60%, at least about 70%, at least about 80%, at least about 90%, or at least about 95% or more.

In various embodiments, the composition is administered in an amount sufficient to reduce intracellular replication of the coronavirus or viral particles thereof.

In various embodiments, the composition is administered at an amount that is further effective to reduce intracellular replication of the coronavirus or viral particles thereof by at least about 10%, at least about 15%, at least about 20%, at least about 25%, at least about 30%, at least about 40%, at least about 50%, at least about 60%, at least about 70%, at least about 80%, at least about 90%, or at least about 95% or more. In various embodiments, the composition is administered at an amount that is further effective to reduce intracellular replication of the coronavirus or viral particles thereof by at least about 50% or more.

In various embodiments, the composition further comprises coenzyme Q10 (CoQ10). In various embodiments, the composition comprises a glycine, an L-glutamate source, L-cystine, L-seleno-methionine and coenzyme Q10 (CoQ10). In various embodiments, the composition further comprises a metallothionein or a fragment thereof. In various embodiments, the method further comprises administering, to a subject in need thereof, a metal chelator. In various embodiments, the method further comprises administering, to a subject in need thereof, an Fe³⁺ chelator, a Zn²⁺ chelator, an Ni²⁺ chelator, or a combination thereof.

In various embodiments, the composition further comprises a therapeutically effective amount of at least one or more of the following: an antiviral agent, an agent for treating fever, or a bronchodilator; wherein each therapeutically effective amount is in a unit dosage form comprising a pharmaceutically acceptable excipient. The method of any of the preceding claims, wherein the effective amount of the composition elevates intracellular concentration of glutathione by at least about 25%, at least about 30%, at least about 40%, at least about 50%, or at least about 60% or more. In various embodiments, the effective amount of the composition elevates intracellular concentration of glutathione by at least about 40%. In various embodiments, the administration of the composition is effective to attain an intracellular concentration of glutathione between 10 μM to 50 μM at 24 hours post-administration. In various embodiments, the administration of the composition is effective to attain an intracellular concentration of glutathione between 20 μM to 40 μM at 48 hours post-administration.

In various embodiments, the composition is administered at a dose that is effective to reduce or inhibit depletion of intracellular glutathione levels in coronavirus or viral particle-infected cells at 24-48 hours post-administration of the composition. In various embodiments, the composition is administered at a dose that is effective to normalize intracellular glutathione levels in SARS-CoV-2 virus- or SARS-CoV-2 viral particle-infected cells to the intracellular glutathione levels in non-infected cells at 24-48 hours post-administration of the composition. In various embodiments, the composition is administered before or after infection with the SARS-CoV-2 virus or viral particles thereof. In various embodiments, the composition is administered after infection with the SARS-CoV-2 virus or viral particles thereof. In various embodiments, the composition is administered about 12 hours to about 96 hours post-infection with the SARS-CoV-2 virus or viral particles thereof. In various embodiments, the composition is administered about 24 hours to about 72 hours post-infection with the SARS-CoV-2 virus or viral particles thereof. In various embodiments, the composition is administered about 48 hours post-infection with the SARS-CoV-2 virus or viral particles thereof.

In various embodiments, the invention relates to a method for reducing the infectivity of severe acute respiratory syndrome coronavirus 2 (SARS-CoV-2) or viral particles thereof, comprising, contacting said SARS-CoV-2 virus or viral particles thereof with a composition that elevates intracellular glutathione. In various embodiments, the virus or viral particle infects a mammalian host cell. In various embodiments, the mammalian host cell is a primate host cell. In various embodiments, the primate host cell is a cell in the lungs, nasal passages, and intestines, any other organs, or combinations thereof. The method of claim 28, wherein the virus infects a biological system selected from a group consisting of a cellular system, a tissue system, an organ system, and an organism.

In various embodiments, the composition is contacted with the SARS-CoV-2 virus or viral particles thereof for a period of about 48 hours. In various embodiments, the infectivity of the SARS-CoV-2 virus or viral particles thereof at least about 10%, at least about 15%, at least about 20%, at least about 25%, at least about 30%, at least about 40%, at least about 50%, at least about 60%, at least about 70%, at least about 80%, at least about 90%, or at least about 95% or more. In various embodiments, the infectivity of the SARS-CoV-2 virus or viral particles thereof is reduced by at least about 60% or more. In various embodiments, the infectivity of the SARS-CoV-2 virus or viral particles thereof is reduced by at least about 80% or more. In various embodiments, the infectivity of the SARS-CoV-2 virus or viral particles thereof is reduced by at least about 90% or more.

In various embodiments, the infectivity of the virus is reduced compared to the infectivity of the virus when contacted with a control composition that does not elevate intracellular glutathione levels. In various embodiments, the control composition does not comprise an amino acid that is glycine, L-cystine, glutamine or glutamate; a selenium source; a metallothionein or a fragment thereof; or the amino acids, the selenium source and the metallothionein or a fragment thereof.

In various embodiments, the invention relates to a method for reducing SARS-CoV-2 viral load in a biological sample comprising cells, comprising contacting the biological sample with an effective amount of a composition that increases intracellular glutathione. In various embodiments, the cells are cells in the lungs, nasal passages, and intestines, any other organs, or combinations thereof. In various embodiments, a SARS-CoV-2 virus or viral particles thereof replication inhibiting amount of the composition is administered.

In various embodiments, the invention relates to a pharmaceutical composition comprising glycine; L-cystine; a glutamate source selected from the group consisting of glutamine and glutamic acid; and a selenium source, for use in treating COVID-19, or reducing the infectivity of the SARS-CoV-2 virus or viral particles thereof, or reducing the SARS-CoV-2 viral load in a subject.

In various embodiments, the composition comprises a glycine, an L-glutamate source, L-cystine, and L-seleno-methionine. In various embodiments, the composition further comprises a coenzyme Q10 (CoQ10). In various embodiments, the composition comprises a glycine, an L-glutamate source, L-cystine, L-seleno-methionine and a coenzyme Q10 (CoQ10). In various embodiments, the composition further comprises a metallothionein or a fragment thereof. In various embodiments, the composition further comprises a metal chelator. In various embodiments, the composition further comprises an Fe³⁺ chelator, a Zn²⁺ chelator, an Ni²⁺ chelator, or a combination thereof. In various embodiments, the composition further comprises a therapeutically effective amount of at least one or more of the following: an antiviral agent, an agent for treating fever, or a bronchodilator; wherein each therapeutically effective amount is in a unit dosage form comprising a pharmaceutically acceptable excipient.

In various embodiments, the invention relates to use of a composition comprising glycine; L-cystine; a glutamate source selected from the group consisting of glutamine and glutamic acid; and a selenium source; optionally together with at least one of the following: coenzyme Q10 (CoQ10); a metallothionein or a fragment thereof a metal chelator; and an Fe³⁺ chelator, a Zn²⁺ chelator, an Ni²⁺ chelator, or a combination thereof, for the manufacture of a medicament for treating COVID-19, or reducing the infectivity of the SARS-CoV-2 virus or viral particles thereof, or reducing the SARS-CoV-2 viral load in a subject. In various embodiments, the composition further comprises the composition comprises a glycine, an L-glutamate source, L-cystine, L-seleno-methionine and coenzyme Q10 (CoQ10). In various embodiments, the composition further comprises the composition further comprises at least one or more of the following: an antiviral agent, an agent for treating fever, and a bronchodilator.

In various embodiments, the invention relates to a kit comprising glycine; L-cystine; a glutamate source selected from the group consisting of glutamine and glutamic acid; and a selenium source; optionally together with at least one of the following: coenzyme Q10 (CoQ10); a metallothionein or a fragment thereof a metal chelator; and an Fe′ chelator, a Zn′ chelator, an Ni′ chelator, or a combination thereof, for treating COVID-19, or reducing the infectivity of the SARS-CoV-2 virus or viral particles thereof, or reducing the SARS-CoV-2 viral load in a subject. In various embodiments, the kit further comprises a glycine, an L-glutamate source, L-cystine, L-seleno-methionine and coenzyme Q10 (CoQ10). In various embodiments, the kit further comprises at least one or more of the following: an antiviral agent, an agent for treating fever, and a bronchodilator.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete.

This disclosure relates to methods for treating and/or preventing coronaviruses (CoV) infections such as the COVID-19 infection caused by SARS-CoV-2. In general, the methods include administering an effective amount of a compound or composition that increases the levels of intracellular glutathione to a subject in need thereof, such as a subject infected by the SARS-CoV-2 virus or at risk of the SARS-CoV-2 virus infection (e.g., a person living in a geographic area in which the SARS-CoV-2 virus is endemic, or in which there is an outbreak). For example, compositions containing glutathione precursors can be administered to inhibit the SARS-CoV-2 virus infectivity and/or the SARS-CoV-2 virus replication.

As described and exemplified herein, the inventors discovered that treatment of cells with a composition containing free-form amino acid precursors of glutathione (FFAAP) increased intracellular glutathione levels in the cells. Surprisingly, FFAAP concomitantly inhibited the replication of the SARS-CoV-2 virus by up to 90% in treated cells in a dose-dependent manner. Additionally, FFAAP also significantly reduced the ability of the SARS-CoV-2 viruses to form plaques, thus demonstrating the ability of FFAAP to inhibit the SARS-CoV-2 viral infectivity. The protective effect against the SARS-CoV-2 virus was observed as early as 24 hours post infection and sustained beyond 72 hours post-infection. Further studies described and exemplified herein demonstrated that total cellular glutathione levels are dramatically decreased in cells infected with the SARS-CoV-2 virus, but that this decrease in glutathione levels was reduced by FFAAP treatment, as was the SARS-CoV-2 virus replication. These results establish the surprising inhibitory effect of FFAAP against the SARS-CoV-2 virus replication and infectivity. Additionally, the results establish that FFAAP treatment relieves the stress inflicted by the SARS-CoV-2 virus infection and replication and exerts a protective effect on treated cells. Without wishing to be bound by any particular theory, it is believed that the SARS-CoV-2-inhibiting effects of FFAAP are due to the direct or indirect actions of elevated glutathione levels induced by FFAAP. Accordingly, methods for the treatment of the SARS-CoV-2 viral diseases, e.g., COVID19, and compositions suitable for use in the methods are disclosed herein.

This disclosure provides a method for treating infections caused by Coronaviruses (CoVs) such as the COVID-19 infection caused by SARS-CoV-2, comprising administering to a subject in need thereof, an effective amount of a composition that increases the levels of intracellular glutathione.

### Scientific Rationale

Metallothioneins. Metallothioneins (MT) belong to a family of cysteine-rich, low molecular weight (MW ranging from 500 to 14000 Da) proteins. They are localized to the membrane of the Golgi apparatus. MTs have the capacity to bind both physiological heavy metals (such as zinc, copper, selenium) and xenobiotic heavy metals (such as cadmium, lead, mercury, silver, arsenic) through the thiol group of its cysteine residues, which represents nearly the 30% of its amino acidic residues. They are thought to play a role in metal detoxification or in the metabolism and homeostasis of metals. MTS are present in a wide variety of eukaryotes including invertebrates, vertebrates, plants, and fungi. See, e.g., Sigel et al. “Metallothioneins and related chelators: Metal Ions in Life Sciences, Cambridge, England: Royal Society of Chemistry (ISBN 1-84755-899-2), which is incorporated by reference in parts pertinent thereto.

Since acute or chronic exposure to heavy metals such as lead, arsenic, mercury or cadmium is implicated in the etiology of a variety of diseases and disorders involving neuromuscular, CNS, cardiovascular, and gastrointestinal effects, metallothioneins have been postulated to play a role in the prevention or alleviation of these conditions. However, a direct and distinct role of metallothioneins in the reduction of incidence and/or treatment of pathogenic diseases, e.g., viral diseases, is unknown.

It was also previously postulated that the aforementioned biological functions of metallothioneins, e.g., proper functioning of neuromuscular, CNS, cardiovascular, and gastrointestinal systems, were only accomplished with low molecular weight metallothionein proteins with a reference range of 500 to 14,000 Daltons (Da). Moreover, synthetic derivatives and precursors of metallothionein were unknown, as most of the earlier work on this area focused on biological isoforms of metallothionein (e.g., MT-I and MT-II) and fragments thereof. See Hillman et al. (U.S. Pat. No. 5,955,428) and Berezin et al. (U.S. Pat. No. 8,618,060), the disclosures in which are incorporated by reference herein in their entirety. Ideally, the metallothionein fragments described herein and in literature have similar or identical biological activity as the full-length proteins (e.g., ability to sequester metal ions).

Genetic delivery of metallothionein isoforms and fragments thereof presents numerous challenges, e.g., technical hurdles associated with the delivery of the gene precisely to target cells; and side effects, such as, infection (due to the vectors used in gene delivery) and tumor development (due to misplaced integration of the gene). Even when delivered properly, the biological metallothionein isoforms and fragments thereof are only located in the membrane of the Golgi apparatus and thus not cytosolically available. Similarly, delivery of complex proteins of metallothionein isoforms is cumbersome, costly, difficult to manufacture in clinical grade and purity, and also face efficacy issues. In this context low molecular weight peptides, e.g., metallothionein fragments, are more bio-available.

Finally, although the biological role of metallothionein has been elucidated in literature, its utility is limited to chelation of metals from samples. There is little, if any evidence to suggest use of metallothionein in the prevention or treatment of pathogenic diseases. In the case of viral diseases where there is no current therapy, for example, infections caused by Coronaviruses (CoVs) such as the COVID-19 infection caused by SARS-CoV-2 virus, novel agents such as metallothionein for the prophylaxis, therapy, chelation therapy and supportive therapy, may fill an unmet need.

Metallothionein analogs in Antiviral Therapy. Embodiments provided herein build upon the recognized role of a selected group of metalloproteins, particularly viral (v) and cellular (c) zinc finger proteins (ZFP) and iron containing proteins in cell proliferation, neovascularization, apoptosis, and viral infection. Along these lines the instant inventor envisioned that disruption of certain metalloproteins by novel pharmacological agents may serve to control and reduce the incidence of many viral diseases. In this regard, embodiments provided herein relate to the potential therapeutic applications of ZFP disrupting agents, zinc chelators and iron chelators in the control of viral diseases. Examples of such proliferative disorders include, but are not limited to, virally transformed cells and cancers relating thereto (e.g., Kaposi's sarcoma, Burkett's lymphoma, adult T-cell leukemia, Merkel cell carcinoma, papilloma-virus induced cancers of cervix, vulva, vagina, penis, anus, etc., and nasopharyngeal carcinoma, etc.).

Due to the central importance and essential functions of viral and cellular zinc-finger proteins, the literature on these topics is now rapidly expanding. Different aspects of ZFP functions, for example, in apoptosis induced by viruses, have been reviewed in recent years. Embodiments of the present invention thus relate to various zinc finger proteins of viruses and cellular zinc finger proteins induced by virus infection, including agents that inhibit their function, in an attempt to critically evaluate some basic biological consequences of manipulating zinc finger proteins.

Conserved Relationship Between ZFP And Viral Replication. All viruses depend on their ability to infect cells and induce them to make more virus particles. If the virus is successful the cells almost invariably die in the process, and that process have been shown to be apoptosis in numerous instances. Other viruses can integrate its DNA in the cellular DNA and remain inactive for long periods. The nucleic acid genome of viruses is always surrounded by a protein shell, denoted capsid, which is composed of nucleocapsid proteins, and some viruses also have a lipid bilayer membrane, termed an envelope, which enclose the nucleocapsid proteins.

Viral ZFPs have been identified in at least two thirds of all viruses studied. See Fernandez-Pol et al, “Essential Viral and Cellular Zinc and Iron Containing Metalloproteins as Targets for Novel Antiviral and Anticancer Agents: Implications for Prevention and Therapy of Viral Disease and Cancer,” Anticancer Research vol. 21:931-958, 2001, which is incorporated by reference in parts pertinent thereto. Examples of families of viruses using metalloproteins such as ZFP, zinc ring proteins or transition metal ion-dependent enzymes for replication, packaging and virulence are Arenaviridae, Reoviridae, Rotaviridae, Retroviridae, Papillomavirinae, Influenza, Adenoviridae, Flaviviridae (Hepatitis C), Herpesviridae, Filoviridae (e.g., Ebola virus and Marburg virus), Pneumovirinae (e.g., RSV), Orthomyxoviridae (Influenza viruses), Coronavirus, etc. Viral ZFP are structural virion proteins essential for viral replication and packaging of the virus inside infected cells. Deletion of zinc finger domains in specific vZFP is lethal to the virus. Since the zinc finger domains of vZFP are essential for viral survival functions, they are conserved throughout evolution and there are no known mutants of the vZFP domain(s). Because the viral zinc finger domain(s) represent indispensable site (s) on the vZFP that can be attacked by one or multiple drugs, vZFP are ideal and primary drug targets for the next generation of antiviral agents.

There are numerous examples of families of viruses that utilize zinc finger proteins, zinc ring proteins and/or transition metal ion-dependent enzymes for specific viral functions. These viral proteins play an essential role in the structure, replication and/or virulence of viruses

Targeting of ZFP for therapy. The National Cancer Institute has identified ZFP as the next target for antiviral drugs (USA Federal Register, 60, No. 154, 1995). Several laboratories are evaluating new antiviral drugs targeted to modify ZFP. These products are targeted towards modification of the amino acid cysteine, which is the binding site for zinc in zinc finger proteins. The present inventor have identified that the cysteine residue of the glutathione molecule, which is synthesized via reconstitution of the precursor components, e.g., glycine, cysteine (as cystine) and glutamate source (for example, glutamine or glutamic acid) confers inhibition of the replication of viruses that rely on such Zn²⁺-binding proteins. Examples of such viruses include, but are not limited to, Ebola viruses (EBV), respiratory syncytial virus (RSV), HIV, HPV, and HSV.

It has been known for many years that the structural and biological properties of viruses can be altered by chelating agents. For example, treatment of rotaviruses with chelating agents such as ethylenediaminetetraacetic acid (EDTA) (10 mM) results in a single-shelled, double-layered, non-infectious viral particles. Moreover, in vitro exposure of various retroviruses to the chelating agents such as EDTA or ethylene glycol tetraacetic acid (EGTA) in millimolar concentrations results in partial disintegration of viral membranes. Thus, disintegration and degradation of retroviruses and rotaviruses can be accomplished by chelating agents.

Similarly, Muller et al. (“Inhibition of Filovirus replication by the zinc finger antiviral protein,” Journal of Virology, 81(5):2391-400, 2007) studied a role of zinc finger antiviral protein (ZAP) against Ebola virus (EBOV) and Marburg virus (MARV). Antiviral effect was observed in cells expressing the N-terminal part of ZAP fused to the product of the zeocin resistance gene (NZAP-Zeo) as well as cells inducibly expressing full-length ZAP. EBOV was inhibited by up to 4 log units, whereas MARV was inhibited between 1 to 2 log units. Transient expression of ZAP decreased the activity of an EBOV replicon system by up to 95%. This inhibitory effect could be partially compensated for by overexpression of L protein. In conclusion, Muller states that the data demonstrate that ZAP exhibits antiviral activity against filoviruses.

Other zinc-binding proteins involved in viral infectivity include, for example, members of the ADAM family of the metalloproteinases. For example, Dolnick et al. (“Ectodomain shedding of the glycoprotein GP of Ebola virus,” The EMBO Journal: 23, 2175-2184, 2004) show that tumor necrosis factor a-converting enzyme (TACE), a member of the ADAM family of zinc-dependent metalloproteases, is involved in the shedding of surface glycoproteins in Ebola viruses. Dolnick further shows that virus-encoded surface glycoproteins are substrates for ADAMs, which cleave them to release them in the blood of virus-infected animals and TACE may play an important role in the pathogenesis of infection by efficiently blocking the activity of virus-neutralizing antibodies. Moreover, inhibitors of zinc-dependent metalloproteinases were shown to inhibit glycoprotein shedding in a concentration-dependent manner. The inhibitory effects were observed with the hydroxamic acid-based inhibitors: BB2516 used at a concentration of 0.5 mM, and GM6001 and MMP-8 inhibitor I used at a concentration of 5 mM. Other inhibitors, such as MMP-3 inhibitor II, CGS-27023A, and TAPI-I, reduced GP shedding at higher concentrations (25-50 mM).

Use Of Chelating Agents To Inhibit Viral Replication. There are several chelating agents that eject the coordinately bound zinc atom from HIV zinc finger proteins. For example, Otzuka et al reported that novel zinc chelators inhibit the DNA-binding activity of zinc finger proteins of HIV. In addition, The Tat trans-activator, is a small protein of 75-130 amino acids, which may form a zinc-finger domain. Since HIV-I lacking Tat replicates poorly and does not cause cytopathic effects, approaches to interfere with Tat may be useful in treating AIDS. The cysteine-rich domain of Tat binds divalent cations, either two Cd2+ or two Zn²⁺ atoms. Whether the cysteine-rich residues form a Zn²⁺ finger or lattice binding pockets for divalent cations is unknown. The pol gene also has a zinc finger amino acid sequence suggesting that chelation chemotherapy may have a role in the treatment of AIDS.

Other research points to the use of competitive inhibition (using peptides that bind to Zn²⁺) as anti-viral agents. See, Hartlieb et al., Journal of Biological Chemistry, 278(43), 40830-40836, 2003.

At least three efficient approaches may be used to design novel classes of inhibitors of viral ZFP activity that directly attack vZFP: 1) disruption of the zinc finger domain by modification of the cysteine residues which are the binding sites for Zn²⁺ in the vZFP, resulting in the ejection of zinc ion; 2) removal of the zinc from the zinc finger moiety by specific chelating agents, which results in inactivation of the vZFP; and 3) specific chelating agents that form a ternary complex at the site of zinc binding on vZFP, resulting in inhibition of the DNA or RNA binding activity of vZFP. Since these antiviral agents attack highly conserved structures in the virus they may circumvent the emergence of drug resistant mutants. Furthermore, the basic mechanisms of action of the novel antivirals (1 through 3, above) may be enhanced in viral disease if the antiviral agents which directly attack metalloproteins of the virus simultaneously attack cellular metalloproteins implicated in the pathogenesis of viral disease. Hence, the novel antivirals may also prove to be effective against cellular zinc finger-containing proteins such as ribosomal ZFP and heat shock proteins which are involved in viral infection. These cellular proteins are induced by the virus for specific viral functions such as replication, propagation, or as an inflammatory response of the cells to the virus.

The specificity of these agents may be due to cellular specificity, in which virally infected cells express cellular and viral ZFPs that are not expressed by normal uninfected cells in their basal or proliferative state. Another primary mode of action of these agents could be receptor specificity, in which vZFP act as receptors for specific zinc ejecting agents, or specific chelating agents which bind to vZFP and form an inactive ternary complex consisting of vZFP-Zn-chelating agent. Thus, vZFP may act as receptors for new agents that can form ternary complexes with vZFP.

Use of Metallothionein Analogs. Embodiments of the present invention provide a solution to the aforementioned problems associated with the delivery and/or use of biological metallothioneins. In one embodiment, there is provided a metallothionein analog comprising a glutathione (GSH) precursor, optionally together with a selenium source. The glutathione precursor comprises (a) L-glycine; (b) L-cystine; and (c) a glutamate source (e.g., glutamine or glutamate), which precursor confers intracellular synthesis of glutathione. See Crum et al. (US patent app. pub. No. 2012-0029082), which is incorporated by reference herein in its entirety. See also US Reissue patent Nos. 39,734 and 42,645, which are incorporated by reference herein. Accordingly, embodiments of the instant invention relate to the use of glutathione formed by the regulated physiological process pathway (trademarked as VITAMIN GSH-S®) as a protective metallothionein analog compound.

In synthesizing glutathione in the body, cysteine, a thiol ammo acid is required. Background research suggests that oral administration of glutathione itself would be ineffective and that prodrugs or precursor therapy would be necessary. Cysteine, or a more bioavailable precursor of cysteine, N-acetyl cysteine (NAC), has been suggested as candidates for precursor therapy. While cysteine and NAC are both, themselves, antioxidants, their presence competes with glutathione for resources in certain reducing (GSH recycling) pathways. Since glutathione is a specific substrate for many reducing pathways, the loading of a host with cysteine or NAC may result in less efficient utilization or recycling of glutathione. Thus, cysteine and NAC are not ideal GSH prodrugs. Thus, while GSH may be degraded, and non-physiologically transported as amino acids, there is a physiological barrier to the importation of intact glutathione. None of the former methods provide a reliable and safe means for increasing intracellular GSH levels.

The compositions and methods of the embodiments described herein therefore provide an improvement over art-known methods for increasing glutathione levels, including importation of intact glutathione molecule into the cytosol using liposome and the like. However, whole glutathione importation into the cell negates the physiologically-perfected synthesis pathway's enzymatic process.

In contrast to the aforementioned suggestions using cysteine or NAC as prodrugs for enhancing cellular GSH levels, embodiments of the present invention relate to alternative methods for elevating levels of physiologically synthesized glutathione and using the glutathione to combat many viral and other pathogenic diseases. In such embodiments, the target system (e.g., cell, tissue, organ or organism) is provided with the components of glutathione (e.g., (a) L-glycine; (b) L-cystine; (c) a glutamate source, e.g., glutamine or glutamate) and optionally the selenium source. The physiologically synthesized glutathione can function as a metallothionein by modulating the optimal reference range for biochemical elemental metals, such as zinc and copper. The metallothionein role can also protect the host from the toxicity of heavy metals (cadmium, lead, silver, arsenic, et al). Unlike biological metallothionein and fragments thereof, the sulfhydryl activity and function of the physiologically synthesized GSH is not limited to molecular weight proteins of 500 to 14,000 daltons, which are located in the membrane of the Golgi apparatus.

In embodiments described herein, the sulfhydryl of a composition previously characterized in RE42,645E can serve a protective function for the host by protecting the body from viral challenges that require elemental metals in order to replicate and proliferate. The compositions of the instant invention, as ion chelators and/or sequestering agents, reduce the infectivity of the pathogenic agents.

Role Of Iron-Binding Proteins In Viral Replication. Growing literature implicates a role of iron-binding proteins in the replication and infection of viruses such as herpes simplex (HIV-1 and HIV-2), Epstein-Barr virus (EBV), varicella-zoster virus (VZV), pseudorabies virus (PRV), and equine herpesvirus type I (EHV-1). Ribonucleotide reductase (RR), which is formed by the association of two non-identical subunits (RI and R2), catalyzes the reduction of ribonucleotide diphosphates to their 2′-deoxy derivatives which is a key intermediate in DNA biosynthesis. There is increasing evidence supporting the essentiality of ribonucleotide reductase (RR) in viral replication. Numerous organisms, including herpes viruses, bacteria, and mammals, encode ribonucleotide reductases the share a number of common characteristics. Two important characteristics of RR are the presence of a stable tyrosyl free radical and the dependency of Fe (III) for catalytic activity. The smaller (R2) subunit contains the iron and tyrosyl radical and the larger (R1) contains thiols which are redox active and provide the hydrogen for nucleotide reduction. The association of R1 and R2 are required for catalytic activity.

Thus, a potential approach for antiviral therapy would be the utilization of peptides that can inhibit enzymatic activity by preventing the association of R1 and R2 subunits. However, since iron is required for catalytic activity a potential, less specific, strategy for antiviral therapy are iron chelating agents, which would deplete iron from the cells, and may have a significant activity against herpes viruses. In 1998, picolinic acid was tested at 3 to 1.5 mM on cultured Human Foreskin (HF) cells infected with HSV-2-strain G and it was found to cause apoptosis of HF infected cells. The specificity of the iron chelators may be cellular specificity rather than viral specificity: infected cells enter apoptosis versus non-infected cells which remain unaffected. See, Romeo et al. (“Intracellular chelation of iron by bipyridyl inhibits DNA virus replication: ribonucleotide reductase maturation as a probe of intracellular iron pools,” Journal of Biological Chemistry, 276(26):24301-8, 2001), which is incorporated by reference herein.

It is relevant to mention that cellular RR is not only an important virulence factor for herpes viruses, but that cellular RR is also involved in the virulence of HIV. It has been suggested that the inhibition of RR with agents such as hydroxyurea could have a possible application in the treatment of AIDS. Giacca et al. have found synergistic antiviral actions of ribonucleotide reductase inhibitors and 3′-azido-3′-deoxythymidine on HIV-1. RR inhibitors reduce the cellular supply of DNA precursors (dNTP) by interfering with their de novo synthesis. A secondary effect is the stimulation of the uptake and phosphorylation of extracellular deoxynucleosides, including their analogs such as 3′-azidothymidine (AZT). Both effects are important to HIV replication, which requires dNTP and is impaired by the triphosphate of AZT. A clear synergism between AZT and RR inhibitors was observed at nontoxic doses.

In vitro studies have shown that glutathione in free form binds iron, particularly Fe²⁺, with high affinity. See, Khan et al. (“Kinetic and spectrophotometric studies of binding of iron(III) by glutathione,” Canadian Journal of Chemistry, 54(20): 3192-3199, 1976), which is incorporated by reference herein in parts pertinent thereto. In accordance therewith, embodiments of the instant invention provide methods of inhibiting pathogenesis of bacterial, viral, or fungal diseases in which iron-binding proteins are implicated in the replication and/or propagation of the pathogenic agents.

Apoptosis. A recent review summarizes the evidence that apoptosis is modulated by intracellular excess or deficiency of Zn²⁺ and presents some mechanism by which Zn²⁺ may control apoptosis (Fernandez-Pol, et al, 2001). The major conclusions are: 1) zinc deficiency, resulting from dietary deprivation or exposure of cultured cells to membrane-permeable Zn²⁺ chelators induces apoptosis; 2) zinc supplementation with Zn²⁺ to the media of cell cultures, can prevent apoptosis; and 3) an intracellular pool of chelatable Zn+ plays a critical role in apoptosis, possibly by modulating the activity of endonucleases. See, Fernandez-Pol et. al., supra.

There is evidence that apoptosis is modulated by intracellular excess or deficiency of Zn²⁺. Fragmentation of DNA and cytolysis are inhibited in certain systems when Zn²⁺ (0.8 mM) is added to the culture medium, It is interesting to note that Ca²⁺/Mg²⁺-dependent endonuclease activity in isolated nuclei was inhibited when Zn²⁺ was added to the medium. These studies are consistent with the hypothesis that Zn²⁺ prevents apoptosis by blocking the activation or inhibiting the activity of Ca²⁺/Mg²⁺-dependent endonuclease. Numerous reports have shown that depletion of intracellular Zn²⁺ by chelation can trigger apoptosis in virally transformed cells. For example, when leukemia cells were exposed to 1,10-phenanthroline, a Zn²⁺/Fe²⁺ chelator, DNA fragmentation and cell death occurred, unless the chelator was neutralized by a transition metal ion added to the medium Similarly, picolinic acid (PA) a Zn²⁺/Fe²⁺ chelator, induces apoptosis in many cells, including leukemia cells by chelating a pool of intracellular Zn²⁺/Fe²⁺, since influx of Zn²⁺/Fe²⁺ prevented apoptosis in the presence of PA, while chelation of Zn²⁺/Fe²⁺ induced apoptosis.

Because Zn²⁺ plays a role in many cellular functions, and because it is a structural component of zinc finger proteins that are essential in cell replication, there are many sites in the apoptotic pathway that can be potentially modulated by zinc and zinc chelators. A number of investigators have shown that apoptosis can be induced if the intracellular levels of Zn²⁺ are reduced using chelators. For example, N,N,N′,N′-tetrakis-2-pyridyl methyl-ethylene diamine (TPEN) added to cultured cells induces apoptosis. These experiments add additional support to the hypothesis that changes in intra- and extracellular zinc can modulate apoptosis. However, none of these chelators are specific for zinc, in fact, some of them are more specific for iron, and they may have chelated a variety of transition metals. Nevertheless, these studies indicate that zinc plays a complex role in a dose and time-dependent manner in apoptosis.

Viruses relevant to human disease such as Smallpox, Ebola virus, Marburg virus, Lassa virus, Papillomavirus, Herpes virus, and Retroviruses, including the AIDS virus, are all capable of inducing apoptosis. Viruses encode genes that both stimulate and suppress apoptotic cell death. These viral proteins interact with cellular pro-apoptotic (death factors) and anti-apoptotic (survival factors). Viral (v) and cellular (c) Zinc finger proteins (ZFP) are involved in apoptotic cell death. A pool of chelatable intracellular Zn²⁺ plays a critical role in viral and cellular apoptosis, possibly by modulating ZFP structure. In virally transformed cells, apoptosis can be induced by intracellular deficiency of Zn²⁺ while normal non-infected cells remain unaffected.

Research has shown that modulation of both v-ZFP and c-ZFP by a class of novel Zn²⁺/Fe²⁺ chelating, broad-spectrum antiviral agents may form ternary complexes with the zinc atoms contained in ZFP. In numerous experiments, research indicates that these wide-spectrum antiviral agents block viral replication and induced apoptosis in virally transformed cells in culture. These agents also interfere with abnormally expressed c-ZFP produced by spontaneously or radiation transformed cells in culture. Thus, these studies provide evidence for a close correlation between interference with ZFP of both viral and cellular origins and apoptosis in transformed but not in normal cells.

The compositions and methods of the invention find utility in the control or treatment of a variety of viruses and viral diseases.

Reducing metal toxicity. In related embodiments, the instant invention provides novel and inventive means for reducing the toxicity caused by metal ions (e.g., due to dysregulation of iron, nickel and/or zinc homeostasis or due to pathogenic conditions) on biological systems. The methods involving contacting the afflicted biological system, which is a cell, a tissue, an organ, or an organism (e.g., a human or a non-human animal) with the aforementioned compositions. Preferably, the compositions comprise glycine, glutamate source (glutamine or glutamic acid) and L-cystine, optionally together with a selenium source (e.g., selenomethionine, selenocysteine, or selenium particles). Further optionally, the compositions may contain additional chelator of Zn²⁺, Fe²⁺ or Ni²⁺, or a combination of such chelators. Preferably, the chelators are bio-compatible and have dissociation constants that are lower than those of proteins which bind to the metal ions (e.g., RR or ZFP). Representative examples of such chelators include, for example, zinc chelators such as N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N-diacetic-N,N′-di-O-propionic (EDPA), etc. and iron chelators include diethylene triamine pentaacetic acid (DETAPAC), dipyridyl, pyridoxal isonicotinoyl hydrazone (PIH), desferrioxamine (DFO), deferiprone (DFP) or deferasirox (DFS). A combination of such chelators may also be employed.

### Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed, as well as the range of values greater than or equal to 1 μM and the range of values less than or equal to 8 μM.

The singular forms “a,” “an,” and “the” include plural referents unless the context clearly dictates otherwise. Thus, for example, reference to an “amino acid” includes a single amino acid as well as two or more of the same or different amino acids; reference to an “excipient” includes a single excipient as well as two or more of the same or different excipients, and the like.

The word “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

An “antioxidant” is a molecule capable of slowing or preventing the oxidation of other molecules. Oxidation is a chemical reaction that transfers electrons from a substance to an oxidizing agent. Oxidation reactions can produce free radicals, which cause oxidative stress and start chain reactions that damage cells. “Oxidative stress” is caused by an imbalance between the production of reactive oxygen and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA. Antioxidants terminate these chain reactions by removing free radical intermediates, and inhibit other oxidation reactions by being oxidized themselves. Examples of antioxidants include, but are not limited to, glutathione, N-acetylcysteine, ascorbic acid, vitamin E, beta-carotene, a polyphenol, flavonoid and an agent that decreases the generation of free radical and non-radical reactive species, including, for example, a CYP2E1 inhibitor, an NAD(P)H oxidase inhibitor or a nitric oxide synthase inhibitor.

“Ascorbic acid” or “vitamin C” refers a monosaccharide antioxidant found in both animals and plants. As one of the enzymes needed to make ascorbic acid has been lost by mutation during human evolution, it must be obtained from the diet and is a vitamin. Most other animals are able to produce this compound in their bodies and do not require it in their diets. In cells, it is maintained in its reduced form by reaction with glutathione, which can be catalyzed by protein disulfide isomerase and glutaredoxins. Ascorbic acid is a reducing agent and can reduce, and thereby neutralize, reactive oxygen species such as hydrogen peroxide. In addition to its direct antioxidant effects, ascorbic acid is also a substrate for the antioxidant enzyme ascorbate peroxidase, a function that is particularly important in stress resistance in plants. Ascorbic acid is present at high levels in all parts of plants and can reach concentrations of 20 millimolar in chloroplasts. Ascorbic acid can be used in combination with iron chelator because it can act as a pro-oxidant in the presence of iron by reducing iron to Fe2+, which would increase the generation of potent oxidants that would damage the nucleic acids.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

A “composition” is intended to mean a combination of an active ingredient (e.g., individual components of the aforementioned metallothionein analogs) and another compound or composition, wherein the second component may be inert (e.g., a carrier) or active (e.g., another metal chelator). In some embodiment, the second component can be, for example, coenzyme Q10 (CoQ10).

The term “chelation” refers to the formation or presence of two or more separate bindings between a polydentate ligand and a single central atom. A “chelant” or “chelator” refers to a chemical that form a soluble and complex molecule with certain metal ions, inactivating the ions so that they cannot normally react with other elements or ions to produce precipitates or scale. A “zinc chelator” refers to a chelator that chelates with zinc ions, e.g., Zn²⁺. An “iron chelator” refers to a chelator that chelates with iron ions, e.g., Fe²⁺/Fe³⁺. Non-limiting examples of zinc chelators include N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N-diacetic-N,N′-di-β-propionic (EDPA), etc. Non-limiting examples of iron chelators include diethylene triamine pentaacetic acid (DETAPAC), dipyridyl, pyridoxal isonicotinoyl hydrazone (PIH), desferrioxamine (DFO), deferiprone (DFP) or deferasirox (DFS) which chelates iron and inhibits metal-catalyzed reactions that produce free radical and non-radical reactive species.

The terms “disease,” “disorder,” and “condition” are used inclusively and refer to any condition mediated at least in part by infection by a pathogenic agent such as viruses, bacteria or the like.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication in vitro or in vivo. “Prophylactically effective” as used herein means the amount of the composition which is sufficient to achieve the desired result, for example, to reduce the incidence of viral infection in a particular subject or a subject population.

As used herein, the term “enhanced” intends a higher level as compared to a control or a prior measurement or value. In one aspect, an enhanced efficacy of an agent or a therapy to reduce or prevent infection of a cell by an RNA virus, which cell is treated with an iron chelator or an antioxidant, is a higher efficacy as compared to the agent or therapy to reduce or prevent infection of the cell by the RNA virus, which cell is not treated with the iron chelator or the antioxidant. In another aspect, it is a higher efficacy as compared to treatment with another, different agent, alone or in combination with the iron chelator or the antioxidant. Enhanced intends an increase by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100%, as compared to a control or prior measurement or value.

The term “glutathione” refers to a cysteine-containing peptide found in most forms of aerobic life. It is not required in the diet and is instead synthesized in cells from its constituent amino acids. Glutathione has antioxidant properties since the thiol group in its cysteine moiety is a reducing agent and can be reversibly oxidized and reduced. In cells, glutathione is maintained in the reduced form by the enzyme glutathione reductase and in turn reduces other metabolites and enzyme systems, such as ascorbate in the glutathione-ascorbate cycle, glutathione peroxidases and glutaredoxins, as well as reacting directly with oxidants. In some organisms glutathione is replaced by other thiols, such as by mycothiol in the Actinomycetes, or by trypanothione in the kinetoplastids. Plasma and liver glutathione concentrations can be raised by oral administration of S-adenosylmethionine (SAMe). Glutathione precursors rich in cysteine include N-acetylcysteine (NAC) and undenatured whey protein, and these supplements have been shown to increase glutathione content within the cell. N-Acetylcysteine, is available both as a drug and as a generic supplement. Alpha Lipoic Acid has also been shown to restore intracellular glutathione. Melatonin has been shown to stimulate a related enzyme, glutathione peroxidase, and silymarin or milk thistle has also demonstrated an ability to replenish glutathione levels. Of all of these methods, the two methods that are the most thoroughly researched for efficacy in raising intracellular glutathione are variants of cysteine. N-acetyl-cysteine, which is a pharmaceutical over the counter drug, and bonded cysteine as is found in the undenatured whey protein nutraceutical, are both proven to be efficacious in raising glutathione values. Also, glutathione can be supplied in the form of glutathione esters.

As used herein, the term “Immune Formulation,” refers to a composition comprising a glutathione precursor and typically also contains a selenium compound, as disclosed in United States Reissue Pat. Nos. 39,734 and 42,645 (each of which is incorporated herein in their entirety). Immune Formulation typically comprises a glutamate source (e.g., glutamic acid or glutamine), cystine and glycine. Immune Formulation may further comprise a selenium source which comprises inorganic selenium compound, e.g., aliphatic metal salts containing selenium in the form of selenite or selenate anions or an organic selenium compound, e.g., selenium cystine, selenium methionine, mono- or di-seleno carboxylic acids comprising about seven to eleven carbon atoms in the chain, or a seleno amino acid chelate. The composition makes available two rate-limiting L-cysteines from the disulfide bond of L-cystine. L-cysteine is rate limiting for biosynthesis of glutathione. When the formulation comprises selenomethionine, the composition further makes available an additional rate-limiting L-cysteine via transsulfuration of the methionine moiety in selenomethionine. Accordingly, Immune Formulation provides the full range of the amino acid precursors needed to form the molecule of glutathione. Physiologically synthesized glutathione maximizes immunological pleiotropy and mechanisms of action without risks of reductive stress.

In one embodiment, Immune Formulation provides the selenium co-factor needed to activate glutathione following its synthesis, and it does not bypass the substrate-specific synthetic enzymes, which provides protection against reductive stress. Additionally, Immune Formulation minimizes risk of vestigiality inherent in importing molecular glutathione, which bypasses the substrate-specific enzymes and the quantitative glutathione regulatory feedback mechanism. The precursor method offers bioavailability advantages by providing individual free form amino acids. Immune Formulation is absorbed immediately into the buccal mucosa. The disulfide bond utilizes the recycling and coupling properties for maintaining and replenishing the rate-limiting L-Cysteine. The free form amino acids provided with Immune Formulation are resistant to degradation and high temperatures; and protected from vigorous agitation and wide pH variation, as compared to high molecular weight whey protein. In one embodiment, Immune Formulation can include coenzyme Q10 (CoQ10).

The “infectivity” of a virus intends the ability of the virus to infect the host. Viral infection is affected by the infectivity, replicative fitness, and the ability of the virus to evade the host's immune response and develop resistance to antivirals.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable” means one that is generally recognized as safe, approved by a regulatory agency of the federal or a state government or listed in the U.S. Pharmacopeia or other generally recognized pharmacopeia for use in animals, and more particularly in humans.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

As used herein, the term “reduced” intends a lower level as compared to a control or a prior measurement or value. In one aspect, a reduced mutation rate of an RNA virus in a cell treated with an iron chelator or an antioxidant refers to a level of mutation rate that is lower than the level of mutation rate of the RNA virus in a cell not treated with the iron chelator or the antioxidant or alternatively, prior to such treatment. In another aspect, it is a lower mutation rate as compared to treatment with another, different agent, alone or in combination with the iron chelator or the antioxidant. Reduced intends a reduction by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100% as compared to a control or prior measurement or value.

The term “restore,” as used herein, refers to a return to an original state or normal state of intracellular glutathione levels after depletion or loss following coronaviruses (CoV) infections, such as the COVID-19 infection caused by SARS-CoV-2.

The term “selenium” is sometimes used hereinafter to include any of the various water-soluble selenium products that can be transported through the mucosal membrane in the practice of this invention. It will be understood, however, that the particular forms of selenium compounds set forth herein are not to be considered limitative. Other selenium compounds, which exhibit the desired activity and are compatible with the other components in the mixture and are non-toxic, can be used in the practice of the invention. Many of them are available commercially.

A “subject” or “patient” is used interchangeably herein, and can be any animal, particularly a vertebrate, preferably a mammal, more preferably a human, and include, but by no means limited to, humans, domestic animals, such as feline or canine subjects, farm animals, such as but not limited to bovine, equine, caprine, ovine, avian and porcine subjects, wild animals (whether in the wild or in a zoological garden), research or laboratory animals, such as mice, rats, rabbits, goats, sheep, pigs, dogs, cats, etc., avian species, such as chickens, turkeys, songbirds, and the like. Besides being useful for human treatment, the present invention is also useful for veterinary treatment of companion mammals, exotic animals and domesticated animals, including mammals, rodents, and the like which is susceptible to viral infection. In one embodiment, the mammals include horses, dogs, and cats.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

As used herein, the terms “treating” refers to the use or administration of a composition, as described herein, including “Immune Formulation,” to treat or prevent a pathologic condition, such as a coronavirus (CoV) infection and COVID-19. Accordingly, treatment can be curative, palliative (e.g., control or mitigate a disease or disease symptoms) or prophylactic (e.g., reduce the frequency of, or delay the onset of a pathologic condition (e.g., fever due to COVID-19 infection) or symptoms in a subject relative to a subject not receiving treatment). This can include reversing, reducing, or arresting the symptoms, clinical signs, and underlying pathology of a condition in a manner to improve or stabilize a subject's condition (e.g., regression of fever).

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., regression of the disease or its clinical symptoms.

“Virus” includes any infectious agent that relies on a “host” for replication. Included in this definition are virions, viral particles, and mature viruses, which are either naturally-occurring or synthetic in nature.

“Vitamin E” is the collective name for a set of eight related tocopherols and tocotrienols, which are fat-soluble vitamins with antioxidant properties. A non-limiting example, .alpha.-tocopherol has been most studied as it has the highest bioavailability, with the body preferentially absorbing and metabolizing this form. .alpha.-tocopherol protects membranes from oxidation by reacting with lipid radicals produced in the lipid peroxidation chain reaction. This removes the free radical intermediates and prevents the propagation reaction from continuing. This reaction produces oxidized .alpha.-tocopheroxyl radicals that can be recycled back to the active reduced form through reduction by other antioxidants, such as ascorbate, retinol or ubiquinol. This is in line with findings showing that .alpha.-tocopherol, but not water-soluble antioxidants, efficiently protects glutathione peroxidase 4 (GPX4)-deficient cells from cell death. Vitamin E is available from dietary sources such as asparagus, avocado, egg, milk, nuts, seeds, spinach, unheated vegetable oil, wheat germ or wholegrain foods.

In order to provide a complete, concise and clear description of the invention, this disclosure includes descriptions of various components, groups of components, ranges and other elements of the broad invention. It is intended that such elements can be variously combined to provide embodiments of the invention. It is also intended that any disclosed features (e.g., substituent, analog, compound, ligand, structure, component) including individual members of any disclosed group, including any sub-ranges or combinations of sub-ranges within the group, may be excluded from the invention or any embodiments of the invention for any reason.

### Compositions for Treatment of Coronaviruses

Compositions suitable for use in the methods disclosed herein can be administered to a subject to increase the level of intracellular glutathione in the subject. Exemplary compositions can contain, for example, reduced glutathione, oxidized glutathione and/or conjugated glutathione, preferably formulated with a suitable delivery system (e.g., liposomes, nanoparticles and the like) to provide for intracellular delivers of the glutathione. Preferred compositions for use in the methods disclosed herein contain FFAAP. More preferred compositions for use in the methods disclosed herein contain FFAAP and a selenium source.

In various embodiments, the composition for use in the treatment of a coronavirus disease comprises a glutathione precursor and a selenium compound formulated in an amount effective to reduce the infectivity of a corona virus. In various embodiments, the glutathione precursor comprises glycine, L-cystine and a glutamate source. In various embodiments the glutamate source is glutamine or glutamic acid. In various embodiments the selenium compound is selenomethionine, selenite, methylselenocysteine or selenium nanoparticles.

The composition used in the methods described herein typically comprises (a) glycine; (b) L-cystine; and (c) a L-glutamate source (e.g., L-glutamine or L-glutamate), each as a free-form amino acid. The composition typically further comprises (d) a selenium source, such as a selenium-containing amino acid (e.g., selenium methionine, selenium cysteine, methylselenocysteine), selenite, or selenium nanoparticles. While each of the a) glycine, (b) L-cystine, (c) glutamate source, and (d) a selenium source are typically components of a single composition, they can be administered in the form of two or more separate compositions if desired.

If desired, the composition can comprise a derivative of one or more of a) glycine, (b) L-cystine, (c) L-glutamate source, and (d) a selenium source. The “derivative” as used herein includes salts, amides, esters, enol ethers, enol esters, acetals, ketals, acids, bases, solvates, hydrates or prodrugs of the free-form amino acids. Such derivatives may be readily prepared by those of skill in this art using known methods for such derivatization. The derivatives suitable for use in the methods described herein may be administered to animals or humans without substantial toxic effects and either are biologically active or are prodrugs.

In one example, the derivatives comprise salts of the amino acids. The term “salt” includes salts derived from any suitable of organic and inorganic counter ions well known in the art and include, by way of example, hydrochloric acid salt or a hydrobromic acid salt or an alkaline or an acidic salt of the aforementioned amino acids.

If desired, the derivative can in addition or alternatively, be solvent addition forms, e.g., a solvate or alcoholate. Solvates contain either stoichiometric or non-stoichiometric amounts of a solvent, and may be formed during the process of crystallization with acceptable solvents such as water, ethanol, and the like. Hydrates are formed when the solvent is water; alcoholates are formed when the solvent is alcohol. Solvates of compounds described herein can be conveniently prepared or formed using routine techniques. The derivative can further comprise amides or esters of the amino acids and/or isomers (e.g., tautomers or stereoisomers) of the amino acids, as desired.

Typically the composition is a mixture of a) glycine, (b) L-cystine, (c) glutamate source, and (d) a selenium source. The term “mixture” refers to a mingling together of two or more substances without the occurrence of a reaction by which they would lose their individual properties. The mixture may contain (a) glycine, (b) L-cystine, (c) glutamate source, and (d) a selenium source in suitable amounts or ratios. For example, the mixture can contain a stoichiometric ratio of L-glutamate:L-cystine:glycine (mole:mole:mole) of e.g., between 4:1:4 to 1:4:1, including, a ratio of 3:1:4, a ratio of 2:1:4, a ratio of 1:1:4, a ratio of 4:1:3, a ratio of 4:1:2, a ratio of 4:1:1, a ratio of 2:1:3, a ratio of 2:1:2, a ratio of 2:1:1, a ratio of 1:1:2, a ratio of 1:2:1, a ratio of 2:2:1, a ratio of 1:2:2, a ratio of 1:3:1, etc. A preferred mixture comprises a stoichiometric ratio of L-glutamate:L-cystine:glycine of about 1:0.5:1 (mole:mole:mole).

Preferably, the composition further comprises a selenium source, e.g., selenomethionine, methylselenocysteine, selenite, or selenium nanoparticles. In such compositions the selenium source is present in an amount sufficient to provide a dose of at least about 0.01 mcg to about 20 mcg of selenium. For example, the selenium source is present in an amount sufficient to provide a dose of at least about 0.02 mcg, about 0.03 mcg, about 0.04 mcg, about 0.05 mcg, about 0.06 mcg, about 0.07 mcg, about 0.08 mcg, about 0.09 mcg, about 0.1 mcg, about 0.2 mcg, about 0.3 mcg, about 0.4 mcg, about 0.5 mcg, about 0.6 mcg, about 0.7 mcg, about 0.8 mcg, about 0.9 mcg, about 1 mcg, about 1.1 mcg, about 1.2 mcg, about 1.3 mcg, about 1.4 mcg, about 1.5 mcg, about 1.6 mcg, about 1.7 mcg, about 1.8 mcg, about 1.9 mcg, about 2.0 mcg, about 2.1 mcg, about 2.2 mcg, about 2.3 mcg, about 2.4 mcg, about 2.5 mcg, about 2.6 mcg, about 2.7 mcg, about 2.8 mcg, about 2.9 mcg, about 3.0 mcg, about 3.1 mcg, about 3.2 mcg, about 3.3 mcg, about 3.4 mcg, about 3.5 mcg, about 3.6 mcg, about 3.7 mcg, about 3.8 mcg, about 3.9 mcg, about 4.0 mcg, about 4.1 mcg, about 4.2 mcg, about 4.3 mcg, about 4.4 mcg, about 4.5 mcg, about 4.6 mcg, about 4.7 mcg, about 4.8 mcg, about 4.9 mcg, about 5.0 mcg, about 5.5 mcg, about 6.0 mcg, about 7 mcg, about 8 mcg, about 9 mcg, about 10 mcg, about 11 mcg, about 12 mcg, about 13 mcg, about 14 mcg, about 15 mcg, about 16 mcg, about 17 mcg, about 18 mcg, about 19 mcg, about 20 mcg or more.

The mixtures of a) glycine, (b) L-cystine, and (c) glutamate source, and optionally (d) a selenium source can be made using any suitable methods. For example, when the composition is in the form of a flowable solid (e.g., granulated, dry powder and the like) the individual components can be micronized, milled or otherwise processed to achieve a desired particle size before or after mixing.

In various embodiments of the invention disclosed herein, a formulation known as “Immune Formulation 200®” is used to treat a coronavirus. As used herein, “Immune Formulation 200®” is used to refer to a composition comprising of glycine, an L-glutamate source, L-cystine, and L-seleno-methionine. (ProImmune Research Institute, Rhinebeck N.Y.).

In other embodiments, a formulation known as “Priothione™” is used in the practice of the claimed invention. Prothione is a pharmaceutical grade capsule comprising glycine, an L-glutamate source, L-cystine, and L-seleno-methionine and Coenzyme Q10.

The compositions used in the methods described herein may comprise a suitable carrier. As used herein, the term “carrier” includes emulsions, suspensions, gels, sols, colloids, and solids that are physiologically and/or pharmaceutically acceptable. Suitable carries are well-known in the art and include, but are not limited to, aqueous solvents, alcohols, particularly polyhydroxy alcohols such as propylene glycol, polyethylene glycol, glycerol, and vegetable and mineral oils. The carriers and/or excipients can be added in various concentrations and combinations to form solutions, suspensions, oil-in-water emulsions or water-in-oil emulsions. If desired, the carrier may be buffered, for example with alkaline buffers, e.g., ammonium buffer, acidic buffers, e.g., ethanoates, citrates, lactates, acetates, etc., or zwitterionic buffers, such as, glycine, alanine, valine, leucine, isoleucine and phenylalanine, Kreb's-Ringer buffer, TRIS, MES, ADA, ACES, PIPES, MOPSO, cholamine chloride, MOPS, BES, TES, HEPES, DIPSO, MOBS, TAPSO, acetamidoglycine, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS, and CABS.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. If desired tonicity adjusting agents can be included, such as, for example, sugars, sodium chloride or combinations thereof. In some embodiments, the composition is isotonic.

The compositions may also include additional ingredients, such as acceptable surfactants, co-solvents, emollients, agents to adjust the pH and osmolarity and/or antioxidants to retard oxidation of one or more component.

In one embodiment, the composition may be formulated as a solution having a concentration of between about 10 μM to about 500 mM, depending on the method of solubilization. Especially, the free form amino acid precursor (FFAAP) may be formulated at a concentration of about 1 mM to about 50 mM, including any value in between, e.g., about 0.5 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 50 mM, about 60 mM, about 70 mM, about 80 mg/m, about 90 mM, about 100 mM, about 150 mM, about 200 mM, about 250 mM, about 300 mM, about 400 mM, or more.

Exemplary formulations within the embodiments described herein may include one or more of the following:

Composition 1. A composition comprising mixture of glycine, an L-glutamate source, L-cystine, and L-seleno-methionine with or without any of the following compositions.

Composition 2. A composition comprising mixture of glycine, an L-glutamate source, L-cystine, L-seleno-methionine, and coenzyme Q10 (CoQ10) with or without any of the following compositions.

Composition 4. A composition comprising a glutathione (GSH) precursor and a selenium source.

Composition 5. The composition in accordance with the foregoing or the following, wherein the glutathione precursor comprises glycine, L-cystine and a glutamate source.

Composition 6. The composition in accordance with the foregoing or the following, wherein the glutathione precursor comprises glycine, L-cystine and glutamate.

Composition 7. The composition in accordance with the foregoing or the following, wherein the glutamine source is glutamate (Glu) or glutamine (Gln).

Composition 8. The composition in accordance with the foregoing or the following, which is a pharmaceutical composition comprising a carrier, a solvent, an excipient, a surfactant or an emollient and optionally further comprising an additional pharmaceutical agent.

Composition 9. The composition in accordance with the foregoing or the following, wherein the selenium source is selenomethionine, selenite, methylselenocysteine, or selenium nanoparticles.

Composition 10. The composition in accordance with the foregoing or the following, further comprising an additional pharmaceutical agent which is N-acetylcysteine, vitamin C, vitamin E, α-lipoic acid, folic acid, vitamins B6 and B12, silibinin, resveratrol or a combination thereof.

Composition 11. The composition in accordance with the foregoing or the following, further comprising a metallothionein or a fragment thereof.

Composition 12. A combination compensating at least two of the aforementioned compositions.

Composition 13. A composition in accordance with the foregoing or the following, which is a pharmaceutical composition.

Composition 14. A composition in accordance with the foregoing or the following, further comprises a metal chelator.

Composition 15. A composition in accordance with the foregoing or the following, which further comprises a Zn²⁺ chelator, a Fe³⁺ chelator, a Ni²⁺ chelator, a combination thereof.

Composition 16. A composition in accordance with the foregoing or the following, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-β-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

Composition 17. A composition in accordance with the foregoing or the following, which further comprises an antiviral selected from the group consisting of abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscamet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, laninamivir, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

The aforementioned compositions and combinations may be formulated to include suitable additives and further pharmaceutical ingredients. Examples of such additives include, but are not limited to, for example, coenzyme Q10 (CoQ10), ubiquinone, 7-keto dehydroepiandosterone (7-keto DHEA), N-acetyl-cysteine, magnesium orotate or a combination thereof. See Hastings et al. (U.S. Pat. No. 6,368,617) and Richardson et al. (U.S. Pat. No. 6,207,190), which are incorporated by reference in parts pertinent thereto.

The compositions can be prepared for administration by any suitable route as oral, parenteral, intranasal, anal, vaginal, topical, subcutaneous and intravenous administration. For oral administration, the composition may be formulated as, for example, a solution, suspension, emulsion, tablet, pill, capsule (e.g., hard or soft shelled gelatin capsules), sustained release formulation, buccal composition, troche, elixir, syrup, wafer, powder or combinations thereof. Oral compositions may be incorporated directly with the food of the diet. Preferred carriers for oral administration comprise inert diluents, edible carriers or combinations thereof.

If desired, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations thereof the foregoing.

Additional formulations which are suitable for other modes of administration include suppositories. Moreover, sterile injectable solutions may be prepared using an appropriate solvent. Generally, dispersions are prepared by incorporating the various sterilized amino acid components into a sterile vehicle which contains the basic dispersion medium and/or the other ingredients. Suitable formulation methods for any desired mode of administration are well known in the art (see, generally, Remington's Pharmaceutical Sciences, 18th Ed. Mack Printing Company, 1990).

### Methods of Treatment

This disclosure provides a method for treating infections caused by Coronaviruses (CoVs) such as the COVID-19 infection caused by SARS-CoV-2, comprising administering to a subject in need thereof, an effective amount of a composition that increases the levels of intracellular glutathione.

In some aspects, the method includes monitoring the subject after the composition that increases the levels of intracellular glutathione is administered, for example to determine efficacy and/or to adjust dosing or dosing interval. Suitable methods to monitor subjects with the SARS-CoV-2 virus infection or COVID-19 are known in the art. The efficacy of treatment using the composition is preferably evaluated by examining the subject's symptoms in a quantitative way, e.g., by noting a decrease in the frequency of adverse symptoms, behaviors, or attacks, or an increase in the time for sustained worsening of symptoms. In a successful course of treatment, the subject's status will improved (i.e., frequency of relapses will have decreased, or the time to sustained progression will have increased). The most common symptoms of SARS-CoV-2 infection can be, but are not limited to, fever, rashes, headaches, joint pain, conjunctivitis (red eyes), muscle pain, nausea etc. SARS-CoV-2 infections are usually mild with symptoms lasting for several days to a week.

In particular applications, the method is for delaying progression, delaying onset, slowing progression, preventing, providing remission, and/or improving symptoms of COVID-19 infection caused by SARS-CoV-2. For example, the method can be for reducing the incidence, duration, or intensity of fever, rashes, headaches, joint pain, conjunctivitis (red eyes), muscle pain, neurological developmental effects, or any combination thereof that are associated with COVID-19 infection caused by SARS-CoV-2, comprising administering to a subject in need thereof, an effective amount of a composition that increases the level of intracellular glutathione.

In the practice of any of the methods disclosed herein, the composition increases the level of intracellular glutathione and can be administered to a subject who is infected by the SARS-CoV-2 virus or who is at risk of infection by the SARS-CoV-2 virus, such as a person living or traveling in an area where the SARS-CoV-2 virus is pandemic. In certain embodiments of the methods, the subject is infected by the SARS-CoV-2 virus. In other embodiments of the methods, the subject is at risk of infection by the SARS-CoV-2 virus. In a particular practice of the methods disclosed herein, the composition that increases the level of intracellular glutathione is administered to the subject at the onset of infection or as soon as practicable after infection, or prior to the expiration of 12-hours post infection (HPI), 24 HPI (1 DPI), 48 HPI (2 DPI), 72 HPI (3 DPI) or 96 HPI (4 DPI).

Preferably, the composition that is administered in any of the methods disclosed herein is an Immune Formulation, such as IMMUNE FORMULATION 200® (mixture of glycine, an L-glutamate source, L-cystine, and L-seleno-methionine, ProImmune Research Institute, Rhinebeck N.Y.). In one embodiment, the composition that is administered in any of the methods disclosed herein is an Immune Formulation, such as IMMUNE FORMULATION 200® (mixture of glycine, an L-glutamate source, L-cystine, and L-seleno-methionine, ProImmune Research Institute, Rhinebeck N.Y.).

The composition that increases the level of intracellular glutathione (e.g., Immune Formulation, IMMUNE FORMULATION 200® (mixture of glycine, an L-glutamate source, L-cystine, and L-seleno-methionine, ProImmune Research Institute, Rhinebeck N.Y.)) can be administered to the subject in need thereof using any suitable or desired mode of administration. For example, the composition can be administered parenterally (e.g., intravenously, intramuscularly, subcutaneously, intraperitonealy, intradermally, intra-articularly, intrathecally, epidurally, intracerebrally), by buccal administration, rectally, topically, transdermally, orally, intranasally, by pulmonary route, intra-opthalmically and retro-orbitally. Parenteral administration (e.g., intravenous administration) can include bolus injection, intermittent infusion, or continuous infusion. Oral and/or buccal administrations are generally preferred.

An effective amount of the composition that increases the level of intracellular glutathione (e.g., Immune Formulation, IMMUNE FORMULATION 200® (mixture of glycine, an L-glutamate source, L-cystine, and L-seleno-methionine, ProImmune Research Institute, Rhinebeck N.Y.)) is administered to a subject in need thereof in the practice of the methods of the invention. An “effective amount” is an amount that is sufficient to achieve the desired effect under the conditions of administration, such as an amount that is sufficient to increase the level of intracellular glutathione, to reduce infectivity or replication of the SARS-CoV-2 virus, to reverse or reduce depletion of intracellular glutathione levels in the SARS-CoV-2 virus-infected cells, to restore intracellular glutathione levels in the SARS-CoV-2 virus-infected cells, or to reduces the progress of, cures or acts palliatively on the SARS-CoV-2 virus infection or COVID-19. The actual amount administered may depend on a variety of factors including the subject's age, general health, body weight, severity of condition, the type of disease being treated, previous or concurrent therapeutic interventions, and other factors. Appropriate amounts to be administered can be determined by a clinician based on these and other considerations. The effective amount can be administered in a single dose or in multiple doses including in a dosing regimen or course of therapy.

When the composition is an Immune Formulation, such as IMMUNE FORMULATION 200® (mixture of glycine, an L-glutamate source, L-cystine, and L-seleno-methionine, ProImmune Research Institute, Rhinebeck N.Y.)) typically an amount that that provides a combined dose of glycine plus L-glutamate source plus L-cysteine from about 0.25 g to about 10.0 g, such as, about 0.50 g, about 0.75 g, about 1.0 g, about 1.25 g, about 1.50 g, about 1.60 g, about 1.75 g, about 2.0 g, about 2.25 g, about 2.5 g, about 2.75 g, about 3.0 g, about 4.0 g, about 5.0 g, about 6.0 g, about 7.0 g, about 8.0 g, about 9.0 g or about 10.0 g or more is administered to a human patient at each administration. If weight adjusted dosing is desired or indicated, an amount that provides a combined dose of glycine plus L-glutamate source plus L-cysteine of about 1 mg/kg/body weight to about 100 g/kg/body weight or more can be administered, for example, about 2 mg/kg/body weight, about 5 mg/kg/body weight, about 10 mg/kg/body weight, about 15 mg/kg/body weight, about 20 mg/kg/body weight, about 25 mg/kg/body weight, about 30 mg/kg/body weight, about 40 mg/kg/body weight, about 50 mg/kg/body weight, about 100 mg/kg/body weight, or about 200 mg/kg/body weight, or more at each administration.

In some examples of the practice of the methods disclosed herein, the effective amount increases intracellular glutathione levels by at least about 20%, at least about 30%, at least about 40%, at least about 50%, at least about 60%, at least about 70%, at least about 80%, at least about 90%, at least about 100%, at least about 120%, at least about 150%, at least about 200%, at least 300% (e.g., compared to pre-treatment level). The effective amount can be an amount that results in an intracellular glutathione concentration of between about 10 μM to about 100 μM, particularly between about 20 μM and about 60 μM, and especially between about 30 μM and about 50 μM, including, a concentration of glutathione of about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 50 μM, about 60 μM, about 70 μM, about 80 μM, about 90 μM, about 100 μM, about 200 μM, about 500 μM, or more. In some embodiments, the effective amount can be an amount that results in an intracellular glutathione concentration of about 1 mM or more, e.g., 1.5 mM, 2.0 mM, 2.5 mM, 3.0 mM, 3.5 mM, 4 mM, 4.5 mM, 5 mM, 6.0 mM, 7.0 mM, 8.0 mM, 9.0 mM, 10 mM, or more. Typically, the increase in intracellular glutathione concentrations is attained about 2 hours to about 72 hours following administration of the composition. Methods of quantitating intracellular glutathione levels, e.g., in terms of concentration or weight ratio (e.g., nano moles/mg of cell protein), are known in the art and exemplified herein. In one embodiment, the concentration values are based on the average calculated volume of the cell type used (e.g., Vero cells having a volume of about ˜0.6 pico liter (pL)/cell) and the glutathione concentration/cell is approximated by first quantifying the total moles of glutathione per unit volume (of media) and then arriving at the intracellular concentration by factoring in the cell volume.

In some examples of the practice of the methods disclosed herein, the effective amount of glutathione reduces infectivity of the SARS-CoV-2 virus by at least about 20%, at least about 30%, at least about 40%, at least about 50%, at least about 60%, at least about 70%, at least about 80%, at least about 90%, at least about 95% or more, compared to controls (e.g., an untreated sample or a sample treated with a buffer alone).

In some examples of the practice of the methods disclosed herein, the effective amount reduces intracellular replication of the SARS-CoV-2 virus by at least about 20%, at least about 30%, at least about 40%, at least about 50%, at least about 60%, at least about 70%, at least about 80%, at least about 90%, at least about 95% or more, compared to controls (e.g., an untreated sample or a sample treated with a buffer alone).

In some examples of the practice of the methods disclosed herein, the effective amount is sufficient to restore intracellular glutathione levels in the SARS-CoV-2 virus-infected cells. Typically, the total intracellular glutathione level, e.g., combined levels of oxidized and reduced glutathione, is restored.

In some examples of the practice of the methods disclosed herein, the effective amount is sufficient to reverse or reduce depletion of intracellular glutathione levels in the SARS-CoV-2 virus-infected cells. As demonstrated in the Examples section, the SARS-CoV-2 virus elicits oxidative stress, resulting in depletion of intracellular glutathione. Treatment of the SARS-CoV-2-infected cells with the compositions can reverse or reduce the oxidative stress. In some embodiments, a reversal or reduction in the SARS-CoV-2-virus-elicited depletion of intracellular glutathione is achieved 4-48 hours post-administration.

In some examples of the practice of the methods disclosed herein, the effective amount is sufficient to produce intracellular concentration of the active components of the composition (e.g., the FFAAP, such as the combined amounts of L-glutamate, L-cystine and glycine) of at least about 2 mM, about 2 mM to about 5 mM, or about 2.5 mM to about 5 mM.

In a further aspect, the disclosure relates to a method for killing or inhibiting the replication of the SARS-CoV-2 virus in a biological sample, comprising contacting the biological sample with an effective amount of a composition that increases the level of intracellular glutathione (e.g., Immune Formulation, IMMUNE FORMULATION 200® (mixture of glycine, an L-glutamate source, L-cystine, and L-seleno-methionine, ProImmune Research Institute, Rhinebeck N.Y.)). In one embodiment, an effective amount of the composition further comprises coenzyme Q10 (CoQ10). As used herein, a “sample” refers to any biological sample that contains the SARS-CoV-2 virus infected cells or the SARS-CoV-2 virus (including, but not limited to, conditioned medium resulting from the growth of cells in cell culture medium, the SARS-CoV-2 virus infected cells, and the like).

In another embodiment, disclosed herein is a method for reducing the SARS-CoV-2 viral load in a biological sample comprising cells, comprising contacting the biological sample with an effective amount of a composition that increases intracellular glutathione.

In another embodiment, disclosed herein is a method for reducing the SARS-CoV-2 viral load in a biological sample comprising blood cells, neural cells or epithelial cells, comprising contacting the biological sample with an effective amount of a composition that increases intracellular glutathione.

### Methods of Reducing the Side Effects of Vaccination

All vaccines, including those for COVID-19, carry the possibility of side effects. Across the globe. The most common side effects following COVID-19 vaccines are fatigue, a fever, headaches, body aches, chills, nausea, diarrhea, and pain at the site of injection. The compositions disclosed herein may also be useful in reducing the side effects of vaccination. In various embodiments, the compositions disclosed herein are delivered at a dosage of an amount that that provides a combined dose of glycine plus L-glutamate source plus L-cysteine from about 0.25 g to about 10.0 g, such as, about 0.50 g, about 0.75 g, about 1.0 g, about 1.25 g, about 1.50 g, about 1.60 g, about 1.75 g, about 2.0 g, about 2.25 g, about 2.5 g, about 2.75 g, about 3.0 g, about 4.0 g, about 5.0 g, about 6.0 g, about 7.0 g, about 8.0 g, about 9.0 g or about 10.0 g or more is administered to a human patient at each administration. If weight adjusted dosing is desired or indicated, an amount that provides a combined dose of glycine plus L-glutamate source plus L-cysteine of about 1 mg/kg/body weight to about 100 g/kg/body weight or more can be administered, for example, about 2 mg/kg/body weight, about 5 mg/kg/body weight, about 10 mg/kg/body weight, about 15 mg/kg/body weight, about 20 mg/kg/body weight, about 25 mg/kg/body weight, about 30 mg/kg/body weight, about 40 mg/kg/body weight, about 50 mg/kg/body weight, about 100 mg/kg/body weight, or about 200 mg/kg/body weight, or more at each administration.

In various embodiments, the composition is delivered twice daily or three times daily. In various embodiments, the composition is delivered once daily. In various embodiments, the dosage is administered at least once or at least twice or at least three times daily, at least two weeks prior to each vaccination. In various embodiments, the dosage is administered at least once, twice or three times daily for at least 1 day, 2 days, 3 days, 4 days, 5 days, 1 week, 2 weeks or 3 weeks after vaccination. In various embodiments, compositions disclosed herein are delivered at a dosage of an amount that that provides a combined dose of glycine plus L-glutamate source plus L-cysteine of about 1.6 mg twice daily prior to vaccination, and the dosage is increased to three times daily immediately after vaccination for at least one week.

In various embodiments, continued administration of the compositions disclosed herein will substantially reduce the side effects of vaccination including fatigue, a fever, headaches, body aches, chills, nausea, diarrhea, and pain at the site of injection.

### Combination Therapies

If desired, the methods disclosed herein can further include administering one or more additional therapeutic agents to the subject in need thereof, such as an antiviral agent, an agent for treating fever, and a bronchodilator. The antiviral agent can be any antiviral agent described in the foregoing sections. The additional therapeutic agent and the composition that increases intracellular glutathione levels are administered so as to provide substantial overlap in their biological activities, and can be administered as components of a single composition or as separate compositions. An effective amount of the additional therapeutic agent is administered and the appropriate amount can be determined based on the subject's age, general health, body weight, severity of condition, the type of disease being treated, previous or concurrent therapeutic interventions, and other factors.

### Kits

Also included within the scope of the present disclosure are kits, e.g., pharmaceutical kits, comprising at least a composition comprising of glycine, L-cystine, a glutamate source selected from the group consisting of glutamine and glutamic acid; and a selenium source and optionally coenzyme Q10 (CoQ10); a metallothionein or a fragment thereof; a metal chelator; and an Fe³⁺ chelator, a Zn²⁺ chelator, an Ni²⁺ chelator, or a combination thereof, for treating COVID-19, or reducing the infectivity of the SARS-CoV-2 virus or viral particles thereof, or reducing the SARS-CoV-2 viral load in a subject. Said kits may also comprise additional agents such as an antiviral agent, an agent for treating fever and a bronchodilator.

### EXAMPLES

The following examples are not meant to be limiting, but are presented to provide further information and support for the present invention. Further, the structures, materials, compositions and methods described herein are intended to be representative of the invention and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1 Treatment of COVID-19 with Immune Formulation 200®

A clinical study is conducted where patients who have tested positive for COVID-19 are each administered a mixture of free-form glycine, an L-glutamate source, L-cystine, and L-seleno-methionine (IMMUNE FORMULATION 200®, ProImmune Research Institute, Rhinebeck, N.Y.). The mixture is administered twice daily at a dose of 1.6 g dissolved in a suitable solution and administered orally.

A screening questionnaire for COVID-19 or other method is used to track daily symptoms. The initial COVID-19 screening questionnaire and follow-up form include the following symptoms for tracking during the course of the illness: loss of sense of smell and/or taste; sore throat; fever, sweats, chills, cough (and whether it was dry or productive with associated shortness of breath); pulse oximetry readings if available, measured both without and with oxygen via nasal cannula, diarrhea, nasal congestion, sneezing, rhinorrhea, conjunctivitis, headaches, myalgias and/or arthralgias, and memory or concentration problems.

### Example 2: Treatment of COVID-19 with Immune Formulation 200® and Coenzyme Q10

A clinical study is conducted where patients who have tested positive for COVID-19 are each administered a mixture of free-form glycine, an L-glutamate source, L-cystine, and L-seleno-methionine (IMMUNE FORMULATION 200®, ProImmune Research Institute, Rhinebeck, N.Y.) and Coenzyme Q10. The IMMUNE FORMULATION 200® (1.6 mg) and the Coenzyme Q10 (6-12 mg) are dissolved in a suitable solution and administered orally.

A screening questionnaire for COVID-19 or other method is used to track daily symptoms. The initial COVID-19 screening questionnaire and follow-up form include the following symptoms for tracking during the course of the illness: loss of sense of smell and/or taste; sore throat; fever, sweats, chills, cough (and whether it was dry or productive with associated shortness of breath); pulse oximetry readings if available, measured both without and with oxygen via nasal cannula, diarrhea, nasal congestion, sneezing, rhinorrhea, conjunctivitis, headaches, myalgias and/or arthralgias, and memory or concentration problems.

These studies demonstrate that by increasing biosynthesis of intracellular levels of glutathione beyond that normally observed remarkably impacted infectious the SARS-CoV-2 virus production in infected mammalian cells.

Further, the experimental design presented above facilitates assessment of some potential role(s) intracellular glutathione against the SARS-CoV-2 virus infection. This is the first study to demonstrate that increased biosynthesis of intracellular glutathione can inhibit the SARS-CoV-2 virus replication. The invention provides for a protective role of IMMUNE FORMULATION 200® in enhancing intracellular biosynthesis of reduced and oxidized glutathione. Additionally, a significant (about 90%) reduction of virus production via treatment with 2-4 mM concentration of intracellular glutathione demonstrates the efficacy of FFAAP to control the SARS-CoV-2 virus infection and highlights the therapeutic potential of FFAAP formulation to treat diseases caused by the SARS-CoV-2 virus in humans and other veterinary animals.

### Example 3: Clinical Observations

Several patients have been treated with Immune Formulation 200® and Coenzyme Q10. Many patients with significant risk factors, including elevated A1C, old age and cardiovascular issues who have been infected with COVID-19 and have been treated with Immune Formulation 200® and Co-Q10, have experienced what appears to be modified disease trajectories. Patients with significant risk factors and who have developed serious symptoms have been treated with Immune Formulation 200® and turned around faster than would be expected from the natural course of disease, with many who appear to be deteriorating, recovering quickly and not requiring hospital care. Multiple other patients without these significant risk factors have also had limited duration of symptoms once Immune Formulation 200® has been administered. In multiple other instances, patients living in the same household as an infected individual have taken Immune Formulation 200® and Coenzyme Q10 and not developed symptomatic infection. Patients in high-risk occupations where high-dose infection is likely have developed only minor symptoms with infection. The dramatic shift in symptom severity that accompanies administration of Immune Formulation 200® has been striking and patients who have been supplementing with Immune Formulation 200® have not developed serious disease.

In clinical practice, patients were given 2 doses×1.6 g/day (total 3.2 gram/day) and others took up to 4 doses×1.6 gram per day of Immune Formulation 200® powder (6.4 gram/day). Due to the novel nature of the Covid-19 pandemic, treatment was initially conservative. The recommended dose was increased from 2 doses daily at 1.6 gram/day to 3 doses a day at 1.6 gram/day for patients who were infected with COVID-19. There were no safety issues observed after 75 patients with the higher dose of gram/day taken over a 4-month period.

### Example 4: A Retrospective Case Review of COVID-19 Patient

IMMUNE FORMULATION 200® was administered to a 71-year-old white male of Greek decent diagnosed with COVID-19. The patient's previous diagnoses included Diabetes Type 2 (uncontrolled, A1C consistently >8 since 2018), hyperlipidemia, hypertension, beta thalassemia, BMI >30, obstructive sleep apnea, chronic atrial fibrillation s/p failed ablation, right bundle branch block, visual field defect from stroke, history of diverticulitis, GERD, history of cerebrovascular accident in 2015, benign prostatic hypertrophy with outflow obstruction, microalbuminuria, 9p21 genotype heterozygous carrier—increased CVD risk, and 4q25 AF genotype heterozygous carrier—increased CVA/atrial fib risk. The patient medications were amlodipine 5 mg tablet—once daily, aspirin 81 mg tablet—once daily, carvedilol 25 mg tablet—once daily, clonidine HCL 0.1 mg tablet—one, three times daily, duloxetine 30 mg capsule—one daily, Januvia 50 mg tablet—one daily, metformin 500 XRmg tablet—one, twice daily, pradaxa 150 mg capsule—one, twice daily, trulicity 1.6 mg/0.5 mL sq pen injector—once weekly, Vitamin D3 5000 IU—one daily, and the IMMUNE FORMULATION 200®.

The patient's son exposed him to COVID-19 on Mar. 23, 2020, and the patient had a few, intermittent doses of IMMUNE FORMULATION 200® leading up to his symptoms. On Apr. 7, 2020, the patient developed a fever of 99.9 and had exhaustion and chest pressure under left side. The patient started to take 1.6 gm IMMUNE FORMULATION 200® daily on this date. On Apr. 9, 2020, the patient's fever had risen to 101.5. On Apr. 10, 2020, symptoms had progressed further and the patient was sent to the ER for increasing chest pain, where he was diagnosed with COVID-19 and had a chest X-ray revealing bilateral interstitial and airspace opacities related to atypical/viral pneumonia. At rest, patient's pulse ox remained above 94%, but dropped to 88% when the patient was ambulated. No fever was reported at the ER, but the patient had been taking Tylenol 650 mg twice daily since Apr. 7, 2020, which could have masked a fever. The patient's IMMUNE FORMULATION 200® dosage was increased to 1.6 gm three times daily. While in the ER, the patient started Proventil HFA 90 mcg/per inhalation aerosol, 2 puffs every 6 hours, as needed. The patient's oxygen saturation remained unchanged and the patient was discharged on Azithromycin 250 mg—2 capsules daily for 3 days, Tessalon pearls 200 mg oral capsule every 8 hours, as needed, and was instructed to return if any signs of shortness of breath or worsening symptoms. IMMUNE FORMULATION 200® was administered every 8 hours continuously. On Apr. 14, 2020, the patient's condition was improving and oxygen saturation remained between 92-95% with no reported shortness of breath. There was a low-grade fever of 99.5 degrees around 2 μm and the patient was taking Tylenol 650 mg daily to resolve the fever. The patient also spoke with his PCP via a telehealth visit and the Azithromycin 250 mg PO QD 5 days was refilled, Ceftin 500 mg bid×7 days was started, and the patient was instructed to call back in a few days and go to ER if any shortness of breath occurred. The patient continued to improve and had complete resolution of symptoms with no relapses or evidence of residual post-COVID-19 syndrome.

Additionally, his wife, age 70, was placed on IMMUNE FORMULATION 200® 1.6 gm daily starting on Apr. 7, 2020 and increased to 1.6 gm twice daily on Apr. 10, 2020, and remained asymptomatic and COVID-19 free to date, despite very close contact with the patient while living together and providing his care.

On Jun. 9, 2021, the patient and his wife had blood work performed. The patient continues to have positive natural immunity with anti-SARS-COV-2-S antibodies, while his wife has negative antibodies. Neither the patient, nor his wife, have received the COVID-19 vaccination. Presently, the patient and his wife continue to take 1.6 gm IMMUNE FORMULATION 200® and CoQ10 supplements daily.

### Example 5: Retrospective Case Review of Patients Who have Received COVID-19 Vaccines

Several patients who have been vaccinated for COVID-19 have experienced little if any side effects from the vaccination when treated with Immune Formulation 200® and Coenzyme Q10 before and after the vaccine. For example:

LS is a 59 year-old female with a history of hypothyroidism. She became fully vaccinated with Pfizer 3/2. She had been taking Immune Formulation 200® 1.6 gm twice daily consistently prior to the vaccine injections and increased to 3 times daily prior to each vaccine and a few days afterwards. She experienced no side effects from either injection with no shifts of blood levels appreciated.

JG is a 52 year-old female with a history of high homocysteine. She completed both of her Moderna vaccines on Feb. 16, 2021. She had been using Immune Formulation 200® consistently at 1.6 gm twice daily prior to each vaccine and increased to 3 times prior to each injection and several days afterwards. She experienced no side effects from either injection with no shifts of blood levels appreciated.

AH is a 48 year old female with a history of arthritis completed both her Pfizer vaccines Apr. 22, 2021. She has been using Immune Formulation 200® 1.6 gm prior to the vaccine and increased to 3 times daily 3 days before and 1 week afterwards. She experienced no side effects not blood work shifts from her vaccine.

In contrast, patients not taking Immune Formulation 200® are known to exhibit adverse reactions upon vaccination for COVID-19.

EK is a 57 year female with a history of hypercholesterolemia and was vaccinated with the Johnson and Johnson vaccine on Mar. 27, 2021 Blood work obtained on Apr. 26, 2021 for her consult revealed significant shifts in her iron levels and estradiol. The patient experienced a postmenopausal bleed post vaccine on Apr. 20, 2021. This was evaluated by endometrial biopsy and no known causation or cancer cells. She was not on Immune Formulation 200® at the time of her vaccination.

SC is a 57 year old female with a history of hypothyroidism and chronic migraines became fully vaccinated with the Pfizer vaccine as of May 2021. Blood work obtained for on Jun. 7, 2021, revealed shifts in her iron levels as well as hormone levels. She experienced excessive hard periods starting May 29, 2021. This was very unusual, and she had not had this problem in the past. She was not on Immune Formulation 200® at the time of her vaccination.

### Example 6: A Phase 2, Proof of Concept Randomized, Double Blind, Placebo Controlled Study to Evaluate the Efficacy and Safety of Prothione™ Capsules for Mild to Moderate Coronavirus Disease 2019 (COVID-19

A clinical study is conducted to assess the efficacy and safety of Prothione™ capsules administered orally twice a day for 30 days in subjects with mild to moderate COVID-19. Three×one gram Prothione™ capsules are administered twice daily for a total daily dosage of: Coenzyme Q10 (12 mg), L-Glutamine (2350.19 mg), Glycine (2350.19 mg), L-cystine (1198.34 mg) and selenomethione (0.034 mg). Safety and clinical efficacy is compared between groups.

Clinical Study Design. A total of 200 subjects are randomized 1:1 and sample size is based on clinical judgment. The study is divided into three phases: (i) Screening Period, (ii) Treatment Period, and Follow-Up Period.

Screening. The Screening Period, which lasts up to 3 days, begins at Visit 1 and includes obtaining signed informed consent, a review of medical and medication history, eligibility evaluation, physical examination and vital signs, clinical symptom score assessment, pulse oxygen saturation, electrocardiogram, and laboratory sample collection for CBC with differentials, red blood cell count with intracellular glutathione, Vitamin B2 and D3, serum 8-OHdG, quantitative c-reactive protein, biochemistry with lipid profile, SARS-COVid-19 viral load, HIV, HCV Ab, HBS Ag, and serum pregnancy (if applicable).

Treatment Period. The Treatment Period includes Visit 2, which is within 3 days of the Screening Visit 1, as well as Visits 3-32, which are 1 day after each previous visit. During Visit 2, there is a review of medical and medication history, physical examination and vital signs, clinical symptom score assessment, pulse oxygen saturation (twice daily, AM and PM), assessment for the requirement of mechanical ventilation, oxygen, and hospital stay, adverse event evaluation, and the random administration of a Prothione™ (three capsules) or placebo orally twice daily. On Visits 3-32, assessments performed are physical examination and vital signs, clinical symptom score assessment, pulse oxygen saturation (twice daily, AM and PM), assessment for the requirement of mechanical ventilation, oxygen, and hospital stay, adverse event evaluation, and administration of Prothione™ or placebo and concomitant medications. Additional assessments of red blood cell count with glutathione, serum 8-OHdG levels, complete blood count with differential, quantitative C-Reactive protein, biochemistry, including fasting lipid profile, Vitamins B2 and D3 levels, SARS-COVID-19 RT-PCR test, HIV viral load in HIV positive subjects, and site specific oropharyngeal cells for intracellular GSH levels are performed multiple times throughout the Treatment Period.

Follow-Up Visit. The Follow-Up Visit is performed 7 days after the last IP administration, and assessments performed are the physical examination and vital signs, clinical symptom score assessment, pulse oxygen saturation, complete blood count with differential, quantitative C-Reactive protein, biochemistry, red blood cell with glutathione, serum 8-OHdG, electrocardiogram, and adverse event evaluation.

Clinical Efficacy. The primary clinical outcome is time (days) to successful clinical recovery from a positive RT-PCR for SARS-COV2 as indicated by two consecutive negative RT-PCR tests measured with two different measurements within a 24-36 hour period. Additional clinical outcome measures include: (i) a change a change in Serum 8-OHdG levels; (ii) increased intracellular glutathione levels in red blood cells; and (iii) a reduction of clinical symptoms (fever, myalgia, dyspnea and cough). Reduction of clinical symptoms may be quantified based on “Time to Clinical Resolution” defined as the time in days from initiation of treatment until the patient reaches a score of 0 in at least three of the following: Fever ([0=none, 1=mild, 2=moderate, and 3=severe]); Myalgia ([0=none, 1=mild, 2=moderate, and 3=severe]); Dyspnea ([0=none, 1=mild, 2=moderate, and 3=severe]); Cough ([0=none, 1=mild, 2=moderate, and 3=severe]). Reduction of clinical symptoms may also be assessed by change in symptom score from baseline. A reduction in mortality is also expected.

Safety. Safety outcomes are also measured. Given that the time between emergence of first symptoms and serious complications is extended in COVID-19, early intervention with highly efficient pro-GSH compounds is a safe approach to treating and/or preventing COVID-19 infection across a large window of time. Safety outcomes measured include (i) incidence of treatment-related adverse events (TEAEs); (ii) incidence and severity of treatment-emergent adverse events (TEAEs); (iii) incidence of serious adverse events (SAEs); (iv) incidence of TEAEs and SAEs leading to discontinuation of study medication; (v) changes in blood chemistry and hematology parameter results; (vi) changes in vital signs including temperature, pulse, respiratory rate, systolic and diastolic blood pressure; (vii) changes in physical examination results; and (viii) changes in electrocardiogram (ECG) results. Given the safety profile of this formulation, incidence of any of these safety measures is considered unlikely.

1. A method for preventing or treating a coronavirus infection or for ameliorating symptoms due to coronavirus infection in a subject in need thereof, comprising administering to the subject an effective amount of a composition that increases intracellular glutathione, wherein said administering prevents or treats infection caused by coronavirus or ameliorates symptoms due to coronavirus infection.

2. The method of claim 1, wherein the composition comprises a glutathione precursor and a selenium compound at an amount effective to reduce infectivity of the coronavirus or viral particles thereof.

3. The method of claim 2, wherein the glutathione precursor comprises glycine, L-cystine and a glutamate source.

4. The method of claim 3, wherein the glutamate source is glutamine or glutamic acid.

5. The method of claim 1, wherein the selenium compound is selenomethionine, selenite, methyl selenocysteine or selenium nanoparticles.

6. The method of claim 1, wherein the infectivity of the coronavirus or viral particles thereof is reduced by at least about 10%, or more.

7. The method of claim 1, wherein the composition is administered in an amount sufficient to reduce intracellular replication of the coronavirus or viral particles thereof.

8. (canceled)

9. (canceled)

10. The method of claim 2, wherein the composition further comprises coenzyme Q10 (CoQ10).

11. The method of any of claim 2, wherein the composition comprises a glycine, an L-glutamate source, L-cystine, L-seleno-methionine and coenzyme Q10 (CoQ10).

12. (canceled)

13. The method of any of claim 1, further comprising administering, to a subject in need thereof, a metal chelator.

14. (canceled)

15. The method of claim 1, wherein the composition further comprises a therapeutically effective amount of at least one or more of the following: an antiviral agent, an agent for treating fever, or a bronchodilator; wherein each therapeutically effective amount is in a unit dosage form comprising a pharmaceutically acceptable excipient.

16. The method of claim 1, wherein the effective amount of the composition elevates intracellular concentration of glutathione by at least about 25%, at least about 30%, at least about 40%, at least about 50%, or at least about 60% or more.

17-51. (canceled)

52. A kit comprising glycine; L-cystine; a glutamate source selected from the group consisting of glutamine and glutamic acid; and a selenium source; optionally together with at least one of the following: coenzyme Q10 (CoQ10); a metallothionein or a fragment thereof; a metal chelator; and an Fe³⁺ chelator, a Zn²⁺ chelator, an Ni²⁺ chelator, or a combination thereof, for treating COVID-19, or reducing the infectivity of the SARS-CoV-2 virus or viral particles thereof, or reducing the SARS-CoV-2 viral load in a subject.

53. The kit of claim 52, comprising a glycine, an L-glutamate source, L-cystine, L-seleno-methionine and coenzyme Q10 (CoQ10).

54. The kit of claim 52, further comprising at least one or more of the following: an antiviral agent, an agent for treating fever, and a bronchodilator.

55. A method for reducing the symptoms of a vaccination against a coronavirus infection or in a subject in need thereof, comprising administering to the subject an effective amount of a composition that increases intracellular glutathione, wherein said administering prevents or treats infection caused by coronavirus or ameliorates symptoms due to coronavirus infection.

56. The method of claim 55, wherein the composition comprises a glutathione precursor and a selenium compound at an amount effective to reduce infectivity of the coronavirus or viral particles thereof.

57. The method of claim 56, wherein the glutathione precursor comprises glycine, L-cystine and a glutamate source.

58. The method of claim 57, wherein the glutamate source is glutamine or glutamic acid.

59. The method of claim 55, wherein the selenium compound is selenomethionine, selenite, methyl selenocysteine or selenium nanoparticles.
